(19) United States
(12) Patent Application Publication
(10) Pub. No.: US 2025/0207168 A1
(43) Pub. Date: Jun. 26, 2025

(54) PRODUCTION CELLS

(71) Applicant: Hoffmann-La Roche Inc., Little Falls, NJ (US)

(72) Inventors: Simon Auslaender, Wolfratshausen (DE); Niels Bauer, Penzberg (DE); Oliver Popp, Penzberg (DE); Tanja Angela Leitner, Huglfing (DE); Marco Boettger, Penzberg (DE); Annette Vogt, München (DE)

(73) Assignee: Hoffmann-La Roche Inc., Little Falls, NJ (US)

(21) Appl. No.: 18/966,005

(22) Filed: Dec. 2, 2024

Related U.S. Application Data
(63) Continuation of application No. PCT/EP2023/064727, filed on Jun. 1, 2023.

(30) Foreign Application Priority Data
Jun. 3, 2022 (EP) 22177168.6

(51) Int. Cl. C12P 21/00; C07K 16/00; C12N 9/02; C12N 15/90

(52) U.S. Cl. CPC C12P 21/00; C12N 9/0071; C12N 15/907; C12Y 114/11004; C07K 16/00; C12N 2510/02

(57) ABSTRACT

Herein is reported a modified mammalian cell wherein the transcriptional activity of a procollagen-lysine,2-oxoglutarate 5-dioxygenases (PLOD) has been eliminated.

### CROSS-REFERENCE TO RELATED APPLICATIONS

This application is a continuation of International Application No. PCT/EP2023/0646727, filed Jun. 1, 2023, which claims benefit of priority to European Patent Application No. 22177168.6 filed Jun. 3, 2022, each of which is incorporated herein by reference in its entirety.

### SEQUENCE LISTING

This application contains a Sequence Listing which has been submitted electronically in XML format and is hereby incorporated by reference in its entirety. Said XML copy, created on Dec. 2, 2024, is named P37684-US_SL.xml and is 78,073 bytes in size.

### FIELD OF THE INVENTION

The current inventions are in the field of recombinant therapeutic protein production. In more detail, herein are reported improved mammalian (e.g., Chinese Hamster Ovary (CHO) or Human Embryonic Kidney (HEK)) production cells as well as their use for the recombinant production of therapeutic proteins, wherein the improvement has been achieved by the reduction of the transcriptional activity of a lysine hydroxylase, especially a lysine 5R-hydroxylase.

### BACKGROUND OF THE INVENTION

In recent years, recombinant polypeptide, such as antibodies and antibody-based molecules, have seen a wide application in the field of disease treatment.

Especially regarding antibodies, after the first wave of antibody-based drugs comprising the naturally occurring IgG format, the next generation of formats is now increasingly seeing molecules, which have substantial modifications and derivations from the classical IgG antibody format. The desire for targeting two or even more antigens sequentially or simultaneously brings up more and more complex antibody formats. With increasing format complexity the number of possible antibody-related side products, such as, e.g., chain mispairings, increases.

One step of a therapeutic antibody on its way to the market is the development of a stable producer cell line, which is characterized by high expression in combination with good quality of the recombinant protein, i.e. low antibody-related side product content.

Xie, Q., et al. (MABS 8 (2016) 371-378) identified in a Chinese hamster ovary (CHO)-expressed, recombinant IgG1 monoclonal antibody a previously unreported+16 Da modification in the sequence of XXXXXXXXXWGQGTLVTVSSASTK (SEQ ID NO: 66) originating from a post-translational hydroxylation of lysine in a consensus sequence (XKG). Although this consensus sequence was present in several locations in the antibody sequence, only a single site on the heavy-chain Fab was found to be modified. They outline that lysine hydroxylation of collagen and proteins containing collagen-like domains occurs in animals and typically serves a functional/structural role as a precursor to crosslinking and O-glycosylation. The hydroxylation of these lysins occurs via the lysyl hydroxylase enzyme, which recognizes the consensus amino acid sequence Xaa-Lys-Gly and converts lysine to 5-hydroxylysine (Hyl). Xie et al. hypothesize that an explanation for the specific modification at position 124 in the heavy chain is that the structure of mAbl near this position may be a more favorable substrate for lysyl hydroxylase, whereas the local structures around other consensus sequence sites are not as readily bound by the enzyme.

EP 1 375 510 reported the gene PLOD2 which codes for telopeptide lysyl hydroxylase (TLH). This enzyme converts telopeptidyl Lys into telopeptidyl Hyl, which can subsequently be converted into hydroxylysine cross-links. Collagen with hydroxylysine cross-links shows a higher resistance to degradation by proteinases than collagen with cross-links derived from hydroxylysine. In one aspect, EP 1 375 510 provides methods and compositions to prepare collagenous materials with varying biodegradation rates by varying the ratio of hydroxylysine cross-links over hydroxylysine cross-links.

U.S. Pat. No. 5,328,913 reported a method of inhibiting cell proliferation and lysyl hydroxylase expression by using minoxidil derivatives. Mia, M. M. and Bank, R. A., reported that the 1κB kinase inhibitor ACHP strongly attenuates TGFβ1-induced myofibroblast formation and collagen synthesis.

WO 2021/028350 reported in situ glycosylated MHC II/CII peptide complexes, i.e., complexes naturally glycosylated during recombinant protein expression in the host cell. The host cells is a genetically engineered cell recombinantly expressing a lysylhydroxylase and a collagen galactosyltransferase, preferably lysylhydroxylase 1 (LH1) and/or lysylhydroxylase 2 (LH2) and collagen galactosyltransferase GLT25D1 and/or GLT25D2. It is outlined that CHO cells commonly used for protein production have been tested and are not able to sufficiently add post-translational modifications at the lysine residues resulting in galactosylhydroxylysine (Gal-Hyl) in collagen or in the MHC II/CII peptide complex described therein. Thus, CHO cells need to be genetically engineered to recombinantly express a lysylhydroxylase and a collagen galactosyltransferase.

Guo, T., et al. (Lab. Invest. 101 (2021) 564-569) reported that PLODs are overexpressed in ovarian cancer and are associated with gap junctions via connexin 43. Especially, they reported that SKOV3 cells stably transfected with shRNA targeting PLOD3 were growing after subcutaneous injection into mice, whereas CAOV3 cells showed significantly suppressed proliferation when PLOD3 was knocked down and no measurable tumor could be spotted. Their conclusion was that expression of PLOD1, PLOD2, and PLOD3 showed trend toward mutual exclusivity, indicating functional non-redundancy in part.

### SUMMARY OF THE INVENTIONS

The subject matter of the herein disclosed inventions is based at least in part on the finding that the knockout of the PLOD gene reduces the level of/abolish the formation of hydroxy lysine in recombinantly produced heterologous proteins.

The subject matter of the herein disclosed inventions is based at least in part on the finding that the knockout of the PLOD gene does not affect overall cell culture performance of the respective modified cell. It has been found that PLOD knockout cell lines have higher biomass formation capacity during cultivation and thereby higher recombinant protein titer compared to cell of the identical genotype except for the PLOD knockout.

The subject matter of the herein disclosed inventions is based at least in part on the finding that the knockout of the PLOD gene does not affect other protein quality attributes. It has been found that CE-SDS and SEC assessment shows comparable or even increased main peak levels and comparable or even reduced side product levels.

The subject matter of the herein disclosed inventions is based at least in part on the finding that the knockout of the PLOD gene reduces filterability issues. Without being bound by this theory, it is assumed that this is due to the fact that collagen cannot be cross-linked any more, especially as CHO cells express many collagen genes in high levels. It has been found that filterability was at least comparable or even improved by reduced filter pressure increase.

The subject matter of the herein disclosed inventions is based at least in part on the finding that the knockout of the PLOD gene reduces cell aggregation during cultivation. Without being bound by this theory, it is assumed that due to the reduction/absence of cross-linked collagen trans binding of different CHO cells via integrin alβ1 cannot be mediated.

In the current invention, the term “knock-out” is used in a functional sense. That is, “knock-out” denotes the functional inactivation of the respective gene. This can be without limitation, e.g., a deletion of the respective gene or parts thereof, an introduction of non-sense mutations resulting in a non-functional gene product after transcription and translation, an introduction of a premature stop codon resulting in the production of non-functional gene products. Thus, the term “knock-out” encompasses different approaches, which result in the reduction or elimination of the function of the respective gene, either by reducing or by eliminating the production of a functional gene product or by truncating or mutating or eliminating the respective gene.

The current invention encompasses the following independent aspects and dependent embodiments:

1. A modified mammalian cell wherein the transcriptional activity of a gene encoding a lysine hydroxylase has been reduced.

2. The modified mammalian cell according to aspect 1, wherein further the transcriptional activity of a gene encoding a prolyl hydroxylase has been reduced.

3. The modified mammalian cell according to any one of aspect 1 or embodiment 2, wherein the lysine hydroxylase is a lysine 5R-hydroxylase.

4. The modified mammalian cell according to any one of aspect 1 or embodiments 2 to 3, wherein the lysine hydroxylase is an oxidoreductase of the Enzyme Class (EC) 1.14.11.

5. The modified mammalian cell according to any one of aspect 1 or embodiments 2 to 4, wherein the lysine hydroxylase is procollagen-lysine,2-oxoglutarate 5-dioxygenases (PLOD).

6. The modified mammalian cell according to any one of aspect 1 or embodiments 2 to 5, wherein the lysine hydroxylase is EC 1.14.11.4.

7. The modified mammalian cell according to any one of embodiments 2 to 6, wherein the prolyl hydroxylase is a prolyl 3-hydroxylase (P3H).

8. The modified mammalian cell according to any one of aspect 1 or embodiments 2 to 7, wherein the lysine hydroxylase is one or two or all of procollagen-lysine,2-oxoglutarate 5-dioxygenase 1 (PLOD1), procollagen-lysine,2-oxoglutarate 5-dioxygenase 2 (PLOD2) and procollagen-lysine,2-oxoglutarate 5-dioxygenase 3 (PLOD3).

9. The modified mammalian cell according to any one of embodiments 2 to 8, wherein the prolyl hydroxylase is prolyl 3-hydroxylase 3 or prolyl 3-hydroxylase 4.

10. The modified mammalian cell according to any one of aspect 1 or embodiments 2 to 9, wherein the transcriptional activity of one or more PLOD gene(s) has been reduced.

11. The modified mammalian cell according to any one of embodiments 2 to 10, wherein the transcriptional activity of one or more P3H gene(s) has been reduced.

12. The modified mammalian cell according to any one of aspect 1 or embodiments 2 to 11, wherein the transcriptional activity of the PLOD1 or/and PLOD2 or/and PLOD3 gene(s) has been reduced.

13. The modified mammalian cell according to any one of embodiments 2 to 12, wherein the transcriptional activity of the P3H3 or/and P3H4 gene(s) has been reduced.

14. The modified mammalian cell according to any one of aspect 1 or embodiments 2 to 13, wherein the transcriptional activity of one or two or all of the PLOD1, PLOD2 and PLOD3 gene has been reduced.

15. The modified mammalian cell according to any one of aspect 1 or embodiments 2 to 14, wherein the transcriptional activity of at least the PLOD2 gene has been reduced so that neither of its splice variants is translated.

16. The modified mammalian cell according to any one of aspect 1 or embodiments 2 to 15, wherein the modified mammalian cell is a modified CHO cell or a modified HEK cell.

17. The modified mammalian cell according to any one of aspect 1 or embodiments 2 to 16, wherein the modified mammalian cell is a modified CHO K1 or a modified HEK293 cell.

18. The modified mammalian cell according to any one of aspect 1 or embodiments 2 to 17, wherein further the transcriptional activity of one or more or all of the MYC gene or/and the BAX gene, or/and the BAK gene, or/and ICAM-1 gene, or/and the SIRT-1 genes has been reduced.

19. The modified mammalian cell according to any one of aspect 1 or embodiments 2 to 18, wherein the reduction is a permanent reduction.

20. The modified mammalian cell according to any one of aspect 1 or embodiments 2 to 19, wherein the reduction is by inactivation.

21. The modified mammalian cell according to any one of aspect 1 or embodiments 2 to 20, wherein the reduction is by gene knockout.

22. The modified mammalian cell according to any one of aspect 1 or embodiments 2 to 21, wherein the reduction is by deleting all or a part of the gene.

23. The modified mammalian cell according to any one of aspect 1 or embodiments 2 to 21, wherein the reduction is by introducing mutations into the gene that result in the production of a non-functional gene product.

24. The modified mammalian cell according to any one of aspect 1 or embodiments 2 to 21, wherein the reduction is by introducing additional stop codons into the gene that result in the production of a non-functional gene product.

25. The modified mammalian cell according to any one of aspect 1 or embodiments 2 to 24, wherein the reduction is by mediated by CRISPR/Cas.

26. The modified mammalian cell according to any one of aspect 1 or embodiments 2 to 25, wherein the modified mammalian cell comprises one or two or more targeted integration landing sites.

27. The modified mammalian cell according to any one of aspect 1 or embodiments 2 to 26, wherein the modified mammalian cell comprises one or two or more targeted integration landing sites each comprising two or three recombinase recognition sequences whereby in case of two or more landing sites the recombinase recognition sequences of each landing site are not compatible with each other.

28. The modified mammalian cell according to any one of aspect 1 or embodiments 2 to 27, wherein the modified mammalian cell comprises one or two or more targeted integration landing sites, whereby one of the targeted integration landing sites comprises the recombinase recognition sequences 3L, LoxFas and L2.

29. The modified mammalian cell according to any one of aspect 1 or embodiments 2 to 28, wherein the modified mammalian cell comprises one or two or more targeted integration landing sites, whereby in one targeted integration landing site one or more nucleic acids that encode a heterologous protein have been integrated.

30. The modified mammalian cell according to any one of aspect 1 or embodiments 2 to 29, wherein at least the reduction of the transcriptional activity of the gene encoding a lysine hydroxylase has been effected after the stable introduction of one or more nucleic acids encoding a heterologous protein.

31. The modified mammalian cell according to any one of aspect 1 or embodiments 2 to 28, wherein the modified mammalian cell comprises one or two or more targeted integration landing sites, whereby in one targeted integration landing site one or more nucleic acids that encode a mono- or multispecific antibody have been integrated.

32. The modified mammalian cell according to any one of aspect 1 or embodiments 2 to 28 and 31, wherein at least the reduction of the transcriptional activity of the gene encoding a lysine hydroxylase has been effected after the stable introduction of one or more nucleic acids encoding a mono- or multispecific antibody.

33. A method for the recombinant production of a heterologous protein comprising the steps of
a) cultivating a modified mammalian cell according to any one of aspect 1 or embodiments 2 to 30 further comprising one or more nucleic acids encoding the heterologous protein in a cultivation medium under conditions suitable for the recombinant expression of the heterologous protein,
b) recovering the heterologous protein from the modified mammalian cell or the cultivation medium,
c) optionally purifying the heterologous protein with one or more chromatography steps, and thereby recombinantly producing the heterologous protein.

34. A method for the recombinant production of a mono- or multispecific antibody comprising the steps of
a) cultivating a modified mammalian cell according to any one of embodiments 31 to 32 in a cultivation medium under conditions suitable for the recombinant expression of the mono- or multispecific antibody,
b) recovering the mono- or multispecific antibody from the modified mammalian cell or the cultivation medium,
c) optionally purifying the mono- or multispecific antibody with one or more chromatography steps, and thereby recombinantly producing the mono- or multispecific antibody.

35. The method according to any one of aspects 33 to 34, wherein the cultivating is for 6 to 16 days.

36. The method according to any one of aspects 33 to 34 and embodiment 35, wherein the cultivating is for 12 to 15 days.

37. The method according to any one of aspects 33 to 34 or embodiments 35 to 36, wherein the cultivating is for about 14 days.

38. The method according to any one of aspects 33 to 34 or embodiments 35 to 37, wherein the cultivating is started with a cell density of 3-5*10\hat{ }5 cells/ml or more.

39. The method according to any one of aspects 33 to 34 or embodiments 35 to 38, wherein the cultivating is started with a cell density of 100*10\hat{ }5 cells/ml or more.

40. The method according to any one of aspects 33 to 34 or embodiments 35 to 39, wherein the cultivating is a fed-batch cultivation.

41. The method according to any one of aspects 33 to 34 or embodiments 35 to 40, wherein the cultivating is a fed-batch cultivating with feeding at least on days 1, 4, 7 and 10.

42. The method according to any one of aspects 33 to 34 or embodiments 35 to 41, wherein the cultivating is a fed-batch cultivating with daily feeding.

43. Use of a modified mammalian cell according to any one of aspect 1 or embodiments 2 to 30 for reducing the amount of lysine hydroxylation in a heterologous protein recombinantly produced with said modified mammalian cell.

44. Use of a modified mammalian cell according to any one of aspect 1 or embodiments 2 to 30 for reducing the amount of hydroxy lysine in a heterologous protein recombinantly produced with said modified mammalian cell.

45. Use of a modified mammalian cell according to any one of aspect 1 or embodiments 2 to 30 for increasing biomass formation in a cultivation of said modified mammalian cell.

46. Use of a modified mammalian cell according to any one of aspect 1 or embodiments 2 to 30 for increasing the titer of a heterologous protein recombinantly produced with said modified mammalian cell.

47. Use of a modified mammalian cell according to any one of aspect 1 or embodiments 2 to 30 for reducing heterologous protein side-product formation in a cultivation of said modified mammalian cell recombinantly expressing said heterologous protein.

48. Use of a modified mammalian cell according to any one of aspect 1 or embodiments 2 to 30 for reducing filter resistance in a heterologous protein preparation recombinantly produced with said modified mammalian cell.

49. Use of a modified mammalian cell according to any one of aspect 1 or embodiments 2 to 30 for reducing cell aggregation during cultivation of said modified mammalian cell.

50. Use of a modified mammalian cell according to any one of embodiments 31 to 32 for reducing the amount of lysine hydroxylation in a mono- or multispecific antibody produced with said modified mammalian cell.

51. Use of a modified mammalian cell according to any one of embodiments 31 to 32 for reducing the amount of hydroxy lysine in a mono- or multispecific antibody produced with said modified mammalian cell.

52. Use of a modified mammalian cell according to any one of embodiments 31 to 32 for increasing biomass formation in a cultivation of said modified mammalian cell.

53. Use of a modified mammalian cell according to any one of embodiments 31 to 32 for increasing the titer of a mono- or multispecific antibody produced with said modified mammalian cell.

54. Use of a modified mammalian cell according to any one of embodiments 31 to 32 for reducing mono- or multispecific antibody side-product formation in a cultivation of said modified mammalian cell recombinantly expressing said mono- or multispecific antibody.

55. Use of a modified mammalian cell according to any one of embodiments 31 to 32 for reducing filter resistance in a mono- or multispecific antibody preparation produced with said modified mammalian cell.

56. Use of a modified mammalian cell according to any one of embodiments 31 to 32 for reducing cell aggregation during cultivation of said modified mammalian cell.

57. Use of the reduction of the transcriptional activity of one or more genes encoding a lysine hydroxylase in a mammalian cell for reducing the amount of lysine hydroxylation in a heterologous protein recombinantly produced with said modified mammalian cell.

58. Use of the reduction of the transcriptional activity of one or more genes encoding a lysine hydroxylase in a mammalian cell for reducing the amount of hydroxy lysine in a heterologous protein recombinantly produced with said modified mammalian cell.

59. Use of the reduction of the transcriptional activity of one or more genes encoding a lysine hydroxylase in a mammalian cell for increasing biomass formation in a cultivation of said modified mammalian cell.

60. Use of the reduction of the transcriptional activity of one or more genes encoding a lysine hydroxylase in a mammalian cell for increasing the titer of a heterologous protein recombinantly produced with said modified mammalian cell.

61. Use of the reduction of the transcriptional activity of one or more genes encoding a lysine hydroxylase in a mammalian cell for reducing heterologous protein side-product formation in a cultivation of said modified mammalian cell recombinantly expressing said heterologous protein.

62. Use of the reduction of the transcriptional activity of one or more genes encoding a lysine hydroxylase in a mammalian cell for reducing filter resistance in a heterologous protein preparation recombinantly produced with said modified mammalian cell.

63. Use of the reduction of the transcriptional activity of one or more genes encoding a lysine hydroxylase in a mammalian cell for reducing cell aggregation during cultivation of said modified mammalian cell.

64. The use according to any one of aspects 57 to 63, wherein further the transcriptional activity of a gene encoding a prolyl hydroxylase has been reduced.

65. The use according to any one of aspects 57 to 63 or embodiment 64, wherein the lysine hydroxylase is a lysine 5R-hydroxylase.

66. The use according to any one of aspects 57 to 63 or embodiment 64 to 65, wherein the lysine hydroxylase is an oxidoreductase of the Enzyme Class (EC) 1.14.11.

67. The use according to any one of aspects 57 to 63 or embodiment 64 to 66, wherein the lysine hydroxylase is procollagen-lysine,2-oxoglutarate 5-dioxygenases (PLOD).

68. The use according to any one of aspects 57 to 63 or embodiment 64 to 67, wherein the lysine hydroxylase is EC 1.14.11.4.

69. The use according to any one of aspects 57 to 63 or embodiment 64 to 68, wherein the prolyl hydroxylase is a prolyl 3-hydroxylase (P3H).

70. The use according to any one of aspects 57 to 63 or embodiment 64 to 69, wherein the lysine hydroxylase is procollagen-lysine,2-oxoglutarate 5-dioxygenase 1 (PLOD1) or/and procollagen-lysine,2-oxoglutarate 5-dioxygenase 2 (PLOD2) or/and procollagen-lysine,2-oxoglutarate 5-dioxygenase 3 (PLOD3).

71. The use according to any one of embodiments 64 to 70, wherein the prolyl hydroxylase is prolyl 3-hydroxylase 3 or prolyl 3-hydroxylase 4.

72. The use according to any one of aspects 57 to 63 or embodiment 64 to 71, wherein the transcriptional activity of one or more PLOD gene(s) has been reduced.

73. The use according to any one of embodiments 64 to 72, wherein the transcriptional activity of one or more P3H gene(s) has been reduced.

74. The use according to any one of aspects 57 to 63 or embodiment 64 to 73, wherein the transcriptional activity of the PLOD1 or/and PLOD2 or/and PLOD3 gene(s) has been reduced.

75. The use according to any one of embodiments 64 to 74, wherein the transcriptional activity of the P3H3 or/and P3H4 gene(s) has been reduced.

76. The use according to any one of aspects 57 to 63 or embodiment 64 to 75, wherein the transcriptional activity of at least the PLOD2 gene has been reduced.

77. The use according to any one of aspects 57 to 63 or embodiment 64 to 76, wherein the transcriptional activity of the PLOD2 gene has been reduced so that neither of its splice variants is translated.

78. The use according to any one of aspects 57 to 63 or embodiment 64 to 77, wherein the mammalian cell is a CHO cell or a modified HEK cell.

79. The use according to any one of aspects 57 to 63 or embodiment 64 to 78, wherein the mammalian cell is a CHO K1 or a HEK293 cell.

80. The use according to any one of aspects 57 to 63 or embodiment 64 to 79, wherein further the transcriptional activity of one or more or all of the MYC gene or/and the BAX gene, or/and the BAK gene, or/and ICAM-1 gene, or/and the SIRT-1 genes has been reduced.

81. The use according to any one of aspects 57 to 63 or embodiment 64 to 80, wherein the reduction is a permanent reduction.

82. The use according to any one of aspects 57 to 63 or embodiment 64 to 81, wherein the reduction is an inactivation.

83. The use according to any one of aspects 57 to 63 or embodiment 64 to 81, wherein the reduction is by deleting all or a part of the gene.

84. The use according to any one of aspects 57 to 63 or embodiment 64 to 81, wherein the reduction is by introducing mutations into the gene that result in the production of a non-functional gene product.

85. The use according to any one of aspects 57 to 63 or embodiment 64 to 81, wherein the reduction is by introducing additional stop codons into the gene that result in the production of a non-functional gene product.

86. The use according to any one of aspects 57 to 63 or embodiment 64 to 85, wherein the reduction is by gene knock-out.

87. The use according to any one of aspects 57 to 63 or embodiment 64 to 86, wherein the reduction is mediated by CRISPR/Cas.

88. The use according to any one of aspects 57 to 63 or embodiment 64 to 87, wherein the mammalian cell comprises one or two or more targeted integration landing sites.

89. The use according to any one of aspects 57 to 63 or embodiment 64 to 88, wherein the mammalian cell comprises one or two or more targeted integration landing sites, whereby in one targeted integration landing site one or more nucleic acids that encode the heterologous protein have been integrated.

90. The use according to any one of aspects 57 to 63 or embodiment 64 to 89, wherein at least the reduction of the transcriptional activity of the gene encoding a lysine hydroxylase has been effected after the stable introduction of one or more nucleic acids encoding the heterologous protein.

91. The use according to any one of aspects 57 to 63 or embodiment 64 to 90, wherein the heterologous protein is a mono- or multispecific antibody.

In addition to the various aspects and embodiments specifically described and claimed, the subject matter of the herein disclosed inventions also encompasses other embodiments having other combinations of the features disclosed and claimed herein. As such, the particular features presented herein, especially presented as aspects or embodiments, can be combined with each other in other manners within the scope of the herein disclosed inventions such that the subject matter of the herein disclosed inventions includes any suitable combination of the individual features or combination of features disclosed herein. The description of specific embodiments has been presented for purposes of illustration and description of the teaching of the invention. It is not intended to be exhaustive or to limit the disclosed subject matter to those embodiments disclosed.

An aspect as used herein relates to independent subject matter of the invention, an embodiment as used herein provides for a more detailed realization of one or more or all independent aspects.

### DETAILED DESCRIPTION

The subject matter of the herein disclosed inventions is based at least in part on the finding that the knockout of the PLOD gene reduces the level of/abolishes the formation of hydroxy lysine in recombinantly produced heterologous proteins.

### GENERAL DEFINITIONS

Useful methods and techniques for carrying out the subject matter of the current inventions are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In certain embodiments, the term about denotes a range of +/−10% of the thereafter following numerical value. In certain embodiments, the term about denotes a range of +/−5% of the thereafter following numerical value.

The terms “comprise(s),” “include(s),” “having,” “has,” “can,” “contain(s)” and variants thereof, as used herein, are intended to be open-ended transitional phrases, terms or words that do not preclude the possibility of additional acts or structures. The term “comprising” also encompasses the term “consisting of”. The present disclosure also contemplates other embodiments “comprising,” “consisting of” and “consisting essentially of,” the embodiments or elements presented herein, whether explicitly set forth or not.

The term “recombinant mammalian cell” as used herein denotes a mammalian cell comprising an exogenous nucleotide sequence capable of expressing a polypeptide. Such recombinant mammalian cells are cells into which one or more exogenous nucleic acid(s) have been introduced, including the progeny of such cells. Thus, the term “a mammalian cell comprising a nucleic acid encoding a heterologous polypeptide” denotes cells comprising an exogenous nucleotide sequence integrated in the genome of the mammalian cell and capable of expressing the heterologous polypeptide. In certain embodiments, the mammalian cell comprising an exogenous nucleotide sequence is a cell comprising an exogenous nucleotide sequence integrated at a single site within a locus of the genome of the host cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different.

The term “recombinant cell” as used herein denotes a cell after genetic modification, such as, e.g., a cell expressing a heterologous polypeptide of interest and that can be used for the production of said heterologous polypeptide of interest at any scale. For example, “a recombinant mammalian cell comprising an exogenous nucleotide sequence” denotes a cell wherein the coding sequences for a heterologous polypeptide of interest have been introduced into the genome of the host cell. For example, “a recombinant mammalian cell comprising an exogenous nucleotide sequence” that has been subjected to recombinase mediated cassette exchange (RMCE) whereby the coding sequences for a polypeptide of interest have been introduced into the genome of the host cell is a “recombinant cell”.

A “mammalian cell comprising an exogenous nucleotide sequence” and a “recombinant cell” are both “transformed cells”. This term includes the primary transformed cell as well as progeny derived therefrom without regard to the number of passages. Progeny may, e.g., not be completely identical in nucleic acid content to a parent cell, but may contain mutations. Mutant progeny that has the same function or biological activity as screened or selected for in the originally transformed cell are encompassed.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated” polypeptide or antibody refers to a polypeptide molecule or antibody molecule that has been separated from a component of its natural environment.

The term “integration site” denotes a nucleic acid sequence within a cell's genome into which an exogenous nucleotide sequence is inserted. In certain embodiments, an integration site is between two adjacent nucleotides in the cell's genome. In certain embodiments, an integration site includes a stretch of nucleotide sequences. In certain embodiments, the integration site is located within a specific locus of the genome of a mammalian cell. In certain embodiments, the integration site is within an endogenous gene of a mammalian cell.

The terms “vector” or “plasmid”, which can be used interchangeably, as used herein, refer to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

As used herein, the term “selection marker” denotes a gene that allows cells carrying the gene to be specifically selected for or against, in the presence of a corresponding selection agent. For example, but not by way of limitation, a selection marker can allow the host cell transformed with the selection marker gene to be positively selected for in the presence of the respective selection agent (selective cultivation conditions); a non-transformed host cell would not be capable of growing or surviving under the selective cultivation conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively be a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells expressing such a molecule can be distinguished from cells not harboring this gene, e.g., by the detection or absence, respectively, of the fluorescence emitted by the encoded polypeptide.

As used herein, the term “operably linked” refers to a juxtaposition of two or more components, wherein the components are in a relationship permitting them to function in their intended manner. For example, a promoter and/or an enhancer is operably linked to a coding sequence if the promoter and/or enhancer acts to modulate the transcription of the coding sequence. In certain embodiments, DNA sequences that are “operably linked” are contiguous and adjacent on a single chromosome. In certain embodiments, e.g., when it is necessary to join two protein encoding regions, such as a secretory leader and a polypeptide, the sequences are contiguous, adjacent, and in the same reading frame. In certain embodiments, an operably linked promoter is located upstream of the coding sequence and can be adjacent to it. In certain embodiments, e.g., with respect to enhancer sequences modulating the expression of a coding sequence, the two components can be operably linked although not adjacent. An enhancer is operably linked to a coding sequence if the enhancer increases transcription of the coding sequence. Operably linked enhancers can be located upstream, within, or downstream of coding sequences and can be located at a considerable distance from the promoter of the coding sequence. Operable linkage can be accomplished by recombinant methods known in the art, e.g., using PCR methodology and/or by ligation at convenient restriction sites. If convenient restriction sites do not exist, then synthetic oligonucleotide adaptors or linkers can be used in accord with conventional practice. An internal ribosomal entry site (IRES) is operably linked to an open reading frame (ORF) if it allows initiation of translation of the ORF at an internal location in a 5′-end-independent manner.

As used herein, the term “exogenous” indicates that a nucleotide sequence does not originate from a specific cell and is introduced into said cell by DNA delivery methods, e.g., by transfection, electroporation, or transformation methods. Thus, an exogenous nucleotide sequence is an artificial sequence wherein the artificiality can originate, e.g., from the combination of subsequences of different origin (e.g. a combination of a recombinase recognition sequence with an SV40 promoter and a coding sequence of green fluorescent protein is an artificial nucleic acid) or from the deletion of parts of a sequence (e.g. a sequence coding only the extracellular domain of a membrane-bound receptor or a cDNA) or the mutation of nucleobases. The term “endogenous” refers to a nucleotide sequence originating from a cell. An “exogenous” nucleotide sequence can have an “endogenous” counterpart that is identical in base compositions, but where the “exogenous” sequence is introduced into the cell, e.g., via recombinant DNA technology.

As used herein, the term “heterologous” indicates that a polypeptide does not originate from a specific cell and the respective encoding nucleic acid has been introduced into said cell by DNA delivery methods, e.g., by transfection, electroporation, or transformation methods. Thus, a heterologous polypeptide is a polypeptide that is artificial to the cell expressing it, whereby this is independent whether the polypeptide is a naturally occurring polypeptide originating from a different cell/organism or is a synthetic polypeptide.

The term “procollagen-lysine,2-oxoglutarate 5-dioxygenase” denotes an oxidoreductase that acts on paired donors, with incorporation or reduction of molecular oxygen, whereby 2-oxoglutarate acts as one donor, and one atom of oxygen is incorporated into each donor. The procollagen-lysine 5-dioxygenase has the EC number 1.14.11.4. Synonymous for “procollagen-lysine,2-oxoglutarate 5-dioxygenase” are “collagen lysine hydroxylase”, “lysyl protocollagen dioxygenase”, “lysine-2-oxoglutarate dioxygenase”, “peptidyl-lysine, 2-oxoglutarate: oxygen oxidoreductase”, “peptidyllysine, 2-oxoglutarate: oxygen 5-oxidoreductase”, “lysyl hydroxylase”, “lysyl 5S-hydroxylase”, “oxygenase, protocollagen lysine, di-”, “LH”, “LLH”, “PLOD”, “procollagen-lysine 1,2-oxoglutarate 5-dioxygenase”, and “Ehlers-Danlos syndrome type VI”.

Three different forms denoted as “1”, “2” and “3” of “procollagen-lysine,2-oxoglutarate 5-dioxygenase” are known. Form 1 has the UniProtKB id Q02809.

The “procollagen-lysine,2-oxoglutarate 5-dioxygenase” is encoded by the PLOD gene. For PLOD2 two splice variant can be expressed (LH2a and LH2b), where LH2b differs from LH2a by incorporating the small exon 13A.

The term “prolyl oxygenase” denotes a proteoglycan, which functions as collagen prolyl hydroxylases. Said prolyl hydroxylases are required for proper collagen biosynthesis, folding and assembly. This protein, like other family members, is thought to reside in the endoplasmic reticulum.

Different forms are known, which are denoted as “1”, “2”, “3” and “4”.

Prolyl 3-hydroxylase 3 (encoded by the P3H3 gene; RefSeq NM_014262), has the enzyme class number 1.14.11.7. Alternative names are “Leprecan-like protein 2” and “Protein B”. The protein has prolyl 3-hydroxylase activity catalyzing the post-translational formation of 3-hydroxyproline in-Xaa-Pro-Gly-sequences in collagens, especially types IV and V (By similarity). The enzymatic reaction is L-proline-[procollagen]+2-oxoglutarate+O (2)=trans-3-hydroxy-L-proline-[procollagen]+succinate+CO (2).

The term “sirtuin-1” denotes an enzyme that is part of signal transduction in mammals, i.e., the NAD-dependent deacetylase sirtuin-1. Sirtuin-1 is encoded by the SIRT-1 gene. Chinese hamster sirtuin-1 has the UniProtKB entry A0A3L7IF96. The effect of SIRT-1 gene inactivation has been described in WO 2020/260327, which is expressly incorporated herein by reference.

The term “myc proto-oncogene protein” denotes a family of regulatory genes encoding transcription factors. This family comprises the proteins c-myc (encoded by the MYC gene), 1-myc (encoded by the MYCL gene), and n-myc (encoded by the MYCN gene). Chinese hamster myc proto-oncogene protein has the genomic location in CHO genome (PICR Genome) (see https://www.ncbi.nlm.nih.gov/assembly/GCF_003668045.3/) of RAZU01000002.1 (8,114,040-8,118,048). The effect of MYC inactivation has been described in WO 2022/063877, which is expressly incorporated herein by reference.

The term “bcl-2-associated X protein” denotes a pro-apoptotic Bcl-2 family member encoded by the BAX gene (also known as Bcl214).

The term “bcl-2 homologous antagonist/killer” denotes a pro-apoptotic Bcl-2 family member encoded by the BAK gene (also known as BCL2 antagonist/killer 1, Bak1, Cdn1, Bcl217, and Bak-like).

The effect of BAX and BAK gene inactivation has been described in WO 2009/151591, which is expressly incorporated herein by reference.

The term “Intercellular Adhesion Molecule 1” denotes a cell surface glycoprotein. Its binding partner are integrins of type CD11a/CD18, or CD11b/CD18. It is also known as CD54 (Cluster of Differentiation 54). In humans, it is encoded by the ICAM-1 gene.

A “reduction of transcriptional activity” with respect to a gene can be achieved by a genetic manipulation, e.g., a substitution, a deletion, an insertion, a duplication, a frameshift, or a translocation, in said gene, whereby one or more functions of the corresponding gene product are reduced or eliminated. In some embodiments, the reduction of transcriptional activity is achieved by introducing a loss-of-function mutation in said gene, i.e., introducing a null mutation that eliminates one or more functions of the corresponding gene product, such as, e.g., a deletion that removes some or all of the coding sequence.

### Antibodies

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

The term “antibody” herein is used in the broadest sense and encompasses various antibody structures, including but not limited to full-length antibodies, monoclonal antibodies, multispecific antibodies (e.g., bispecific antibodies), and antibody-antibody fragment-fusions as well as combinations thereof.

The term “native antibody” denotes naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a heavy chain variable region (VH) followed by three heavy chain constant domains (CH1, CH2, and CH3), whereby between the first and the second heavy chain constant domain a hinge region is located. Similarly, from N- to C-terminus, each light chain has a light chain variable region (VL) followed by a light chain constant domain (CL). The light chain of an antibody may be assigned to one of two types, called kappa (κ) and lambda (λ), based on the amino acid sequence of its constant domain.

The term “full-length antibody” denotes an antibody having a structure substantially similar to that of a native antibody. A full-length antibody comprises two full length antibody light chains each comprising in N- to C-terminal direction a light chain variable region and a light chain constant domain, as well as two full length antibody heavy chains each comprising in N- to C-terminal direction a heavy chain variable region, a first heavy chain constant domain, a hinge region, a second heavy chain constant domain and a third heavy chain constant domain. In contrast to a native antibody, a full-length antibody may comprise further immunoglobulin domains, such as e.g. one or more additional scFvs, or heavy or light chain Fab fragments, or scFabs conjugated to one or more of the termini of the different chains of the full length antibody, but only a single fragment to each terminus. These conjugates are also encompassed by the term full-length antibody.

The “class” of an antibody refers to the type of constant domains or constant region, preferably the Fc-region, possessed by its heavy chains. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 8, E, y, and u, respectively.

The term “heavy chain constant region” denotes the region of an immunoglobulin heavy chain that contains the constant domains, i.e., the CH1 domain, the hinge region, the CH2 domain and the CH3 domain. In certain embodiments, a human IgG constant region extends from Ala118 to the carboxyl-terminus of the heavy chain (numbering according to Kabat EU index). However, the C-terminal lysine (Lys447) of the constant region may or may not be present (numbering according to Kabat EU index). The term “constant region” denotes a dimer comprising two heavy chain constant regions, which can be covalently linked to each other via the hinge region cysteine residues forming inter-chain disulfide bonds.

The term “heavy chain Fc-region” denotes the C-terminal region of an immunoglobulin heavy chain that contains at least a part of the hinge region (middle and lower hinge region), the CH2 domain and the CH3 domain. In certain embodiments, a human IgG heavy chain Fc-region extends from Asp221, or from Cys226, or from Pro230, to the carboxyl-terminus of the heavy chain (numbering according to Kabat EU index). Thus, an Fc-region is smaller than a constant region but essentially identical thereto with respect to the C-terminal portion. However, the C-terminal lysine (Lys447) of the heavy chain Fc-region may or may not be present (numbering according to Kabat EU index). The term “Fc-region” denotes a dimer comprising two heavy chain Fc-regions, which can be covalently linked to each other via the hinge region cysteine residues forming inter-chain disulfide bonds.

The constant region, more precisely the Fc-region, of an antibody (and the constant region likewise) is directly involved in complement activation, Clq binding, C3 activation and Fc receptor binding. While the influence of an antibody on the complement system is dependent on certain conditions, binding to Clq is caused by defined binding sites in the Fc-region. Such binding sites are known in the state of the art and described e.g., by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g., L234, L235, D270, N297, E318, K320, K322, P331 and P329 (numbering according to EU index of Kabat). Antibodies of subclass IgG1, IgG2 and IgG3 usually show complement activation, Clq binding and C3 activation, whereas IgG4 do not activate the complement system, do not bind Clq and do not activate C3.

An “Fc-region of an antibody” is a term well known to the skilled artisan and defined based on the papain cleavage of antibodies.

The term “monoclonal antibody” as used herein refers to an antibody obtained from a population of substantially homogeneous antibodies, i.e., the individual antibodies constituting the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies may be made by a variety of techniques, including but not limited to the hybridoma method, recombinant DNA methods, phage-display methods, and methods utilizing transgenic animals containing all or part of the human immunoglobulin loci.

The term “valent” as used within the current application denotes the presence of a specified number of binding sites in an antibody. As such, the terms “bivalent”, “tetravalent”, and “hexavalent” denote the presence of two binding sites, four binding sites, and six binding sites, respectively, in an antibody.

A “monospecific antibody” denotes an antibody that has a single binding specificity, i.e., specifically binds to one antigen. Monospecific antibodies can be prepared as full-length antibodies or antibody fragments (e.g., F(ab′) 2) or combinations thereof (e.g., full length antibody plus additional scFv or Fab fragments). A monospecific antibody does not need to be monovalent, i.e., a monospecific antibody may comprise more than one binding site specifically binding to the one antigen. A native antibody, for example, is monospecific but bivalent.

A “multispecific antibody” denotes an antibody that has binding specificities for at least two different epitopes on the same antigen or two different antigens. Multispecific antibodies can be prepared as full-length antibodies or antibody fragments (e.g., F(ab′) 2 bispecific antibodies) or combinations thereof (e.g., full length antibody plus additional scFv or Fab fragments). A multispecific antibody is at least bivalent, i.e., comprises two antigen binding sites. In addition, a multispecific antibody is at least bispecific. Thus, a bivalent, bispecific antibody is the simplest form of a multispecific antibody. Engineered antibodies with two, three or more (e.g., four) functional antigen binding sites have also been reported (see, e.g., US 2002/0004587).

In certain embodiments of all aspects and embodiments of the subject matter of the herein disclosed inventions, the cell according to the inventions expresses an antibody. In certain embodiments, the antibody is a multispecific antibody, e.g., at least a bispecific antibody. In certain embodiments, one of the binding specificities is for a first antigen and the other is for a different second antigen. In certain embodiments, multispecific antibodies may bind to two different epitopes of the same antigen. Multispecific antibodies may also be used to localize cytotoxic agents to cells, which express the one or more antigens.

Multispecific antibodies can be prepared as full-length antibodies or antibody-antibody fragment-fusions.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g.,

Kostelny, S. A., et al., J. Immunol. 148 (1992) 1547-1553); using the common light chain technology for circumventing the light chain mis-pairing problem (see, e.g., WO 98/50431); using specific technology for making bispecific antibody fragments (see, e.g., Holliger, P., et al., Proc. Natl. Acad. Sci. USA 90 (1993) 6444-6448); and preparing trispecific antibodies as described, e.g., in Tutt, A., et al., J. Immunol. 147 (1991) 60-69).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting Fab” or “DAF” (see, e.g., US 2008/0069820 and WO 2015/095539).

Multi-specific antibodies may also be provided in an asymmetric form with a domain crossover, i.e., by exchanging the VH/VL domains (see, e.g., WO 2009/080252 and WO 2015/150447), the CH1/CL domains (see, e.g., WO 2009/080253) or the complete Fab arms (see e.g., WO 2009/080251, WO 2016/016299, also see Schaefer et al., Proc. Natl. Acad. Sci. USA 108 (2011) 1187-1191, and Klein at al., MAbs 8 (2016) 1010-1020) in one or more binding arms of the same antigen specificity. In certain embodiments of all aspects and embodiments of the subject matter of the herein disclosed inventions, the cell according to the inventions expresses a multispecific antibody comprising a Cross-Fab fragment. The term “Cross-Fab fragment” denotes a Fab fragment, wherein either the variable regions or the constant regions of the heavy and light chain are exchanged. A Cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab heavy chain fragment and cognate light chain pairing. See, e.g., WO 2016/172485.

The antibody or fragment may also be a multispecific antibody as described in WO 2009/080254, WO 2010/112193, WO 2010/115589, WO 2010/136172, WO 2010/145792, or WO 2010/145793.

The antibody or fragment thereof may also be a multispecific antibody as disclosed in WO 2012/163520.

Various further molecular formats for multispecific antibodies are known in the art and can be produced using a cell according to the current inventions (see e.g., Spiess et al., Mol. Immunol. 67 (2015) 95-106).

Bispecific antibodies are generally antibody molecules that specifically bind to two different, non-overlapping epitopes on the same antigen or to two epitopes on different antigens.

In certain embodiments of all aspects and embodiments of the subject matter of the herein disclosed inventions, the cell according to the current inventions expresses a complex (multispecific) antibody selected from the group of complex (multispecific) antibodies consisting of:
a full-length antibody with domain exchange (i.e., a multispecific IgG antibody comprising a first Fab fragment and a second Fab fragment, wherein in the first Fab fragment
a) only the CH1 and CL domains are replaced by each other (i.e., the light chain of the first Fab fragment comprises a VL and a CH1 domain and the heavy chain of the first Fab fragment comprises a VH and a CL domain);
b) only the VH and VL domains are replaced by each other (i.e., the light chain of the first Fab fragment comprises a VH and a CL domain and the heavy chain of the first Fab fragment comprises a VL and a CH1 domain); or
c) the CH1 and CL domains are replaced by each other and the VH and VL domains are replaced by each other (i.e., the light chain of the first Fab fragment comprises a VH and a CH1 domain and the heavy chain of the first Fab fragment comprises a VL and a CL domain); and
wherein the second Fab fragment comprises a light chain comprising a VL and a CL domain, and a heavy chain comprising a VH and a CH1 domain;
the full-length antibody with domain exchange may comprises a first heavy chain including a CH3 domain and a second heavy chain including a CH3 domain, wherein both CH3 domains are engineered in a complementary manner by respective amino acid substitutions, in order to support heterodimerization of the first heavy chain and the modified second heavy chain, e.g., as disclosed in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, or WO 2013/096291 (incorporated herein by reference)););
a full-length antibody with domain exchange and additional heavy chain C-terminal binding site (BS) (i.e., a multispecific IgG antibody comprising
a) one full length antibody comprising two pairs each of a full length antibody light chain and a full length antibody heavy chain, wherein the binding sites formed by each of the pairs of the full length heavy chain and the full length light chain specifically bind to a first antigen, and
b) one additional Fab fragment, wherein the additional Fab fragment is fused to the C-terminus of one heavy chain of the full length antibody, wherein the binding site of the additional Fab fragment specifically binds to a second antigen, wherein the additional Fab fragment specifically binding to the second antigen i) comprises a domain crossover such that a) the light chain variable domain (VL) and the heavy chain variable domain (VH) are replaced by each other, or b) the light chain constant domain (CL) and the heavy chain constant domain (CH1) are replaced by each other, or ii) is a single chain Fab fragment);
a one-armed single chain antibody
(i.e., an antibody comprising a first binding site that specifically binds to a first epitope or antigen and a second binding site that specifically binds to a second epitope or antigen, whereby the individual chains are as follows
   light chain (variable light chain domain+light chain kappa constant domain)
   combined light/heavy chain (variable light chain domain+light chain constant domain+peptidic linker+variable heavy chain domain+CH1+Hinge+CH2+CH3 with knob mutation)
   heavy chain (variable heavy chain domain+CH1+Hinge+CH2+CH3 with hole mutation));
a two-armed single chain antibody
(i.e., an antibody comprising a first binding site that specifically binds to a first epitope or antigen and a second binding site that specifically binds to a second epitope or antigen, whereby the individual chains are as follows
   combined light/heavy chain 1 (variable light chain domain+light chain constant domain+peptidic linker+variable heavy chain domain+CH1+Hinge+CH2+CH3 with hole mutation)
   combined light/heavy chain 2 (variable light chain domain+light chain constant domain+peptidic linker+variable heavy chain domain+CH1+Hinge+CH2+CH3 with knob mutation));
a common light chain bispecific antibody
(i.e., an antibody comprising a first binding site that specifically binds to a first epitope or antigen and a second binding site that specifically binds to a second epitope or antigen, whereby the individual chains are as follows
light chain (variable light chain domain+light chain constant domain)
   heavy chain 1 (variable heavy chain domain+CH1+Hinge+CH2+CH3 with hole mutation)
   heavy chain 2 (variable heavy chain domain+CH1+Hinge+CH2+CH3 with knob mutation));
   a T-cell bispecific antibody (TCB) (i.e., a full-length antibody with additional heavy chain N-terminal binding site with domain exchange comprising
   a first and a second Fab fragment, wherein each binding site of the first and the second Fab fragment specifically bind to a first antigen,
   a third Fab fragment, wherein the binding site of the third Fab fragment specifically binds to a second antigen, and wherein the third Fab fragment comprises a domain crossover such that the variable light chain domain (VL) and the variable heavy chain domain (VH) are replaced by each other, and
   an Fc-region comprising a first Fc-region polypeptide and a second Fc-region polypeptide,
wherein the first and the second Fab fragment each comprise a heavy chain fragment and a full-length light chain,
wherein the C-terminus of the heavy chain fragment of the first Fab fragment is fused to the N-terminus of the first Fc-region polypeptide,
wherein the C-terminus of the heavy chain fragment of the second Fab fragment is fused to the N-terminus of the variable light chain domain of the third Fab fragment and the C-terminus of the CH1 domain of the third Fab fragment is fused to the N-terminus of the second Fc-region polypeptide);
an antibody-multimer-fusion (i.e., a multimeric fusion protein comprising
(a) an antibody heavy chain and an antibody light chain, and
(b) a first fusion polypeptide comprising in N- to C-terminal direction a first part of a non-antibody multimeric polypeptide, an antibody heavy chain CH1 domain or an antibody light chain constant domain, an antibody hinge region, an antibody heavy chain CH2 domain and an antibody heavy chain CH3 domain, and a second fusion polypeptide comprising in N- to C-terminal direction the second part of the non-antibody multimeric polypeptide and an antibody light chain constant domain if the first polypeptide comprises an antibody heavy chain CH1 domain or an antibody heavy chain CH1 domain if the first polypeptide comprises an antibody light chain constant domain,
wherein
(i) the antibody heavy chain of (a) and the first fusion polypeptide of (b), (ii) the antibody heavy chain of (a) and the antibody light chain of (a), and (iii) the first fusion polypeptide of (b) and the second fusion polypeptide of (b) are each independently of each other covalently linked to each other by at least one disulfide bond,
wherein
the variable domains of the antibody heavy chain and the antibody light chain form a binding site specifically binding to an antigen).

The “knobs into holes” dimerization modules and their use in antibody engineering are described in Carter P.; Ridgway J. B. B.; Presta L. G.: Immunotechnology, Volume 2, Number 1, February 1996, pp. 73-73 (1).

The CH3 domains in the heavy chains of an antibody can be altered by the “knob-into-holes” technology, which is described in detail with several examples in e.g., WO 96/027011, Ridgway, J. B., et al., Protein Eng. 9 (1996) 617-621; and Merchant, A. M., et al., Nat. Biotechnol. 16 (1998) 677-681. In this method, the interaction surfaces of the two CH3 domains are altered to increase the heterodimerization of these two CH3 domains and thereby of the polypeptide comprising them. Each of the two CH3 domains (of the two heavy chains) can be the “knob”, while the other is the “hole”. The introduction of a disulfide bridge further stabilizes the heterodimers (Merchant, A. M., et al., Nature Biotech. 16 (1998) 677-681; Atwell, S., et al., J. Mol. Biol. 270 (1997) 26-35) and increases the yield.

The mutation T366W in the CH3 domain (of an antibody heavy chain) is denoted as “knob-mutation” or “mutation knob” and the mutations T366S, L368A, Y407V in the CH3 domain (of an antibody heavy chain) are denoted as “hole-mutations” or “mutations hole” (numbering according to Kabat EU index). An additional inter-chain disulfide bridge between the CH3 domains can also be used (Merchant, A. M., et al., Nature Biotech. 16 (1998) 677-681) e.g. by introducing a S354C mutation into the CH3 domain of the heavy chain with the “knob-mutation” (denotes as “knob-cys-mutations” or “mutations knob-cys”) and by introducing a Y349C mutation into the CH3 domain of the heavy chain with the “hole-mutations” (denotes as “hole-cys-mutations” or “mutations hole-cys”) (numbering according to Kabat EU index).

The term “domain crossover“ ” as used herein denotes that in a pair of an antibody heavy chain VH—CH1 fragment and its corresponding cognate antibody light chain, i.e., in an antibody Fab (fragment antigen binding), the domain sequence deviates from the sequence in a native antibody in that at least one heavy chain domain is substituted by its corresponding light chain domain and vice versa. There are three general types of domain crossovers, (i) the crossover of the CH1 and the CL domains, which leads by the domain crossover in the light chain to a VL-CH1 domain sequence and by the domain crossover in the heavy chain fragment to a VH-CL domain sequence (or a full length antibody heavy chain with a VH-CL-hinge-CH2-CH3 domain sequence), (ii) the domain crossover of the VH and the VL domains, which leads by the domain crossover in the light chain to a VH-CL domain sequence and by the domain crossover in the heavy chain fragment to a VL-CH1 domain sequence, and (iii) the domain crossover of the complete light chain (VL-CL) and the complete VH—CH1 heavy chain fragment (“Fab crossover”), which leads to by domain crossover to a light chain with a VH—CH1 domain sequence and by domain crossover to a heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH and VL domains are “replaced by each other” it is referred to the domain crossover mentioned under item (iii). Bispecific antibodies including domain crossovers are reported, e.g., in WO 2009/080251, WO 2009/080252, WO 2009/080253, WO 2009/080254 and Schaefer, W., et al, Proc. Natl. Acad. Sci. USA 108 (2011) 11187-11192. Such antibodies are generally termed CrossMab.

In certain embodiments of all aspects and embodiments of the subject matter of the herein disclosed inventions, the cell according to the inventions expresses a multispecific antibody comprising at least one Fab fragment including either a domain crossover of the CH1 and the CL domains, or a domain crossover of the VH and the VL domains, or a domain crossover of the VH—CH1 and the VL-VL domains. In multispecific antibodies with domain crossover, the Fabs specifically binding to the same antigen(s) are constructed to be of the same domain sequence. Hence, in case more than one Fab with a domain crossover is contained in the multispecific antibody, said Fab(s) specifically bind to the same antigen.

A “humanized” antibody refers to an antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., the CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “recombinant antibody”, as used herein, denotes all antibodies (chimeric, humanized and human) that are prepared, expressed, created or isolated by recombinant means, such as using a cell according to the current inventions. This includes antibodies isolated from recombinant cells such as NSO, HEK, BHK, amniocytes, or CHO cells modified according to the current inventions.

As used herein, the term “antibody fragment” refers to a molecule other than an intact antibody that comprises a portion of an intact antibody and that binds to the same epitope on the same antigen to which the intact antibody binds, i.e., it is a functional fragment. Examples of antibody fragments include but are not limited to Fv; Fab; Fab′; Fab′-SH; F(ab′) 2; bispecific Fab; diabodies; linear antibodies; single-chain antibody molecules (e.g., scFv or scFab).

### Recombinant Methods and Compositions

One aspect of the subject matter of the herein disclosed inventions is a method of making a heterologous polypeptide or protein using a cell according to the current invention. Said method comprises the steps of culturing a cell according to the current invention comprising one or more nucleic acid(s) encoding the heterologous polypeptide or protein, under conditions suitable for expression of the heterologous polypeptide or protein, and recovering the heterologous polypeptide or protein from the cell (or cell culture medium), and optionally purifying the heterologous polypeptide or protein with one or more chromatography steps.

Antibodies may be produced using recombinant methods and compositions, e.g., as described in U.S. Pat. No. 4,816,567. For these methods, one or more isolated nucleic acid(s) encoding an antibody are provided.

In one aspect of the subject matter of the herein disclosed inventions, a method of making an antibody is provided, wherein the method comprises culturing a cell according to the current invention comprising one or more nucleic acid(s) encoding the antibody, under conditions suitable for expression of the antibody, and optionally recovering the antibody from the cell (or cell culture medium).

For recombinant production of a heterologous polypeptide or protein, such as, e.g., an antibody, nucleic acids encoding the heterologous polypeptide or protein are isolated and inserted into one or more vectors for further cloning and/or expression in a cell according to the current invention. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Generally, for the recombinant large-scale production of a heterologous polypeptide or protein of interest, such as e.g., a therapeutic antibody, a cell stably expressing and secreting said heterologous polypeptide or protein is required. This cell is termed “recombinant cell” or “recombinant production cell” and the process used for generating such a cell is termed “cell line development”.

In the first step of the cell line development process, a suitable host cell, such as, e.g., a CHO cell modified according to the current inventions, is transfected with a nucleic acid sequence suitable for expression of said heterologous polypeptide or protein of interest. In a second step, a cell stably expressing the heterologous polypeptide or protein of interest is selected based on the co-expression of a selection marker, which had been co-transfected with the nucleic acid encoding the heterologous polypeptide or protein of interest.

A nucleic acid encoding a heterologous polypeptide or protein, i.e., the coding sequence, is denoted as a structural gene. Such a structural gene is pure coding information. Thus, additional regulatory elements are required for expression thereof. Therefore, a structural gene is integrated in a so-called expression cassette. The minimal regulatory elements needed for an expression cassette to be functional in a mammalian cell are a promoter functional in said mammalian cell, which is located upstream, i.e., 5′, to the structural gene, and a polyadenylation signal sequence functional in said mammalian cell, which is located downstream, i.e., 3′, to the structural gene. The promoter, the structural gene and the polyadenylation signal sequence are arranged in an operably linked form.

In case the heterologous protein of interest is a heteromultimeric protein that is composed of different (monomeric) polypeptides, such as e.g., an antibody or a complex antibody format, not only a single expression cassette is required but a multitude of expression cassettes differing from each other in the contained structural gene, i.e., at least one expression cassette for each of the different (monomeric) polypeptides of the heteromultimeric protein.

For example, a full-length antibody is a heteromultimeric protein comprising two copies of a light chain as well as two copies of a heavy chain. Thus, a full-length antibody is composed of two different polypeptides. Therefore, two expression cassettes are required for the expression of a full-length antibody, one for the light chain and one for the heavy chain. If, for example, the full-length antibody is a bivalent bispecific antibody, i.e., the antibody comprises two different binding sites specifically binding to two different antigens, the two light chains as well as the two heavy chains are also different from each other. Thus, such a bivalent bispecific, full-length antibody is composed of four different polypeptides and therefore, four expression cassettes are required.

The expression cassette(s) required for the expression of the heterologous polypeptide or protein of interest is (are) in turn integrated into one or more so called “expression vector(s)”. An “expression vector” is a nucleic acid providing all required elements for the amplification of said vector in prokaryotic cells as well as those required for the expression of the comprised structural gene(s) in a mammalian cell. Typically, an expression vector comprises a prokaryotic plasmid propagation unit, e.g. for E. coli, comprising an origin of replication, and a prokaryotic selection marker, as well as a eukaryotic selection marker, and further the expression cassettes required for the expression of the structural gene(s) of interest. An “expression vector“ ” is a transport vehicle for the introduction of expression cassettes into a modified mammalian cell according to the current inventions.

As outlined in the previous paragraphs, the more complex the heterologous polypeptide or protein of interest is, the higher is also the number of required different expression cassettes. Inherently with the number of expression cassettes also the total size, i.e., the number of base pairs, of the nucleic acid to be integrated into the genome of the host cell increases. Concomitantly also the size of the expression vector increases. However, there is a practical upper limit to the size of a vector in the range of about 15 kbps above which handling and processing efficiency profoundly drops. This issue can be addressed by using two or more expression vectors. Thereby the expression cassettes are split between different expression vectors each comprising only some of the expression cassettes resulting in a reduction of the size (number of bp) of the individual vectors.

Cell line development (CLD) for the generation of a recombinant cell expressing a heterologous polypeptide or protein, such as e.g., a multispecific antibody, employs either random integration (RI) or targeted integration (TI) of the nucleic acid(s) comprising the respective expression cassettes required for the expression and production of the heterologous polypeptide or protein of interest.

Using RI, in general, several vectors or fragments thereof integrate into the cell's genome at the same or different loci. Thus, the number as well as the ratio of integrated expression cassettes cannot be controlled.

Using TI, in general, a defined number of the different expression cassettes is integrated at a predetermined “hot-spot” in the cell's genome.

Suitable cells for the expression of an (glycosylated) heterologous polypeptide or protein, such as an antibody, are generally derived from multicellular organisms such as, e.g., vertebrates.

### Host Cells

Any mammalian cell can be modified according to the subject matter of the herein disclosed inventions. This application of the modification according to the subject matter of the herein disclosed inventions is independent from the cell being an adherent growing cell or a cell growing in suspension as well as from the integration method, i.e., for RI as well as TI. Any mammalian cell can be used.

Examples of mammalian cells that can be modified according to the subject matter of the herein disclosed inventions are human amniocyte cells (e.g. CAP-T cells as described in Woelfel, J. et al., BMC Proc. 5 (2011) P133); monkey kidney cells (CV1); monkey kidney CV1 cells transformed by SV40 (COS-7); human embryonic kidney cells (HEK293 or HEK293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor cells (MMT 060562); TRI cells (as described, e.g., in Mather, J. P. et al., Annals N.Y. Acad. Sci. 383 (1982) 44-68); MRC 5 cells; and FS4 cells.

Especially useful mammalian cells to be modified according to the subject matter of the herein disclosed inventions include Chinese hamster ovary (CHO) cells, including DHFR-CHO cells (Urlaub, G. et al., Proc. Natl. Acad. Sci. USA 77 (1980) 4216-4220); as well as myeloma cells such as YO, NSO and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In certain embodiments of all aspects and embodiments of the subject matter of the herein disclosed inventions, the mammalian cell modified according to the current inventions or used in a method or use according to the current inventions is a Chinese Hamster Ovary (CHO) cell (e.g. CHO K1, CHO DG44, etc.), a Human Embryonic Kidney (HEK) cell, a lymphoid cell (e.g., YO, NSO, Sp2/0 cell), or a human amniocyte cells (e.g. CAP-T, etc.). In one preferred embodiment of the subject matter of the herein disclosed inventions, the mammalian cell modified according to the current inventions or used in a method according to the current inventions is a CHO cell or a HEK cell.

In certain embodiments of the subject matter of the herein disclosed inventions, the current inventions relates to modified mammalian cells, e.g., CHO cells, where the expression of one or more mammalian cell endogenous products (e.g., host cell proteins), is reduced or eliminated. For example, but not by way of limitation, methods for reducing or eliminating endogenous product expression in a mammalian cell include: (1) modification of a gene coding for the endogenous product or component thereof, e.g., by introducing a deletion, insertion, substitution, or combination thereof into the gene; (2) reducing or eliminating the transcription and/or stability of the mRNA encoding the endogenous product or a component thereof; and (3) reducing or eliminating the translation of the mRNA encoding the endogenous product or a component thereof. In certain embodiments of the subject matter of the herein disclosed inventions, the reduction or elimination of protein expression is obtained by targeted genome editing. For example, CRISPR/Cas9-based genome editing can be employed to modify one or more target genes, resulting in the reduction or elimination of expression of the gene (or genes) targeted for editing.

In certain embodiments of the subject matter of the herein disclosed inventions, the mammalian cell endogenous product targeted for reduced or eliminated expression is selected based on its role in promoting lysine hydroxylation. As lysine hydroxylation can result in the formation of unwanted by- or side-products, reducing or eliminating expression of such endogenous proteins can positively influence culture yield and purity. For example, but not by way of limitation, the endogenous mammalian cell protein selected based on its role in promoting lysine hydroxylation is PLOD. In certain embodiments of the subject matter of the herein disclosed inventions, the mammalian cells of the subject matter of the herein disclosed inventions exhibit reduced or eliminated expression of PLOD. In certain embodiments of the subject matter of the herein disclosed inventions, the mammalian cells of the present disclosure exhibit reduced or eliminated expression of PLOD 1 or/and PLOD2 or/and PLOD3.

In certain embodiments of the subject matter of the herein disclosed inventions, the mammalian cell comprises a further endogenous product targeted for reduced or eliminated expression.

In certain embodiments of the subject matter of the herein disclosed inventions, one or more further mammalian cell endogenous products targeted for reduced or eliminated expression are selected based on their role in promoting apoptosis. As apoptosis can decrease culture viability and productivity, reducing or eliminating expression of such proteins can positively influence culture viability and productivity. For example, but not by way of limitation, the mammalian cell protein selected based on its role in promoting apoptosis is BCL2 Associated X, Apoptosis Regulator (BAX) or BCL2 Antagonist/Killer 1 (BAK). In certain embodiments of the subject matter of the herein disclosed inventions, the mammalian cells of the present inventions further exhibit reduced or eliminated expression of BAX. In certain embodiments of the subject matter of the herein disclosed inventions, the mammalian cells of the present invention further exhibit reduced or eliminated expression of BAK. In certain embodiments of the subject matter of the herein disclosed inventions, the mammalian cells of the present invention further exhibit reduced or eliminated expression of BAX and BAK.

In certain embodiments of the subject matter of the herein disclosed inventions, the further mammalian cell endogenous product targeted for reduced or eliminated expression is selected based on its role in promoting clumping and/or aggregation during cell culture. When mammalian cells are used for production of a recombinant product of interest, such clumping and/or aggregation during cell culture can lead to reduced product titers due to the negative impact of clumping and/or aggregation on mammalian cell viability. For example, but not by way of limitation, the further mammalian cell endogenous product selected based on its role in promoting clumping and/or aggregation during cell culture is Intercellular Adhesion Molecule 1 (ICAM-1). In certain embodiments of the subject matter of the herein disclosed inventions, the mammalian cells of the present inventions further exhibit reduced or eliminated expression of ICAM-1.

In certain embodiments of the subject matter of the herein disclosed inventions, one or more of the further mammalian cell endogenous products targeted for reduced or eliminated expression are selected based on their role in promoting inefficient cell growth. Mammalian cells express many endogenous products that are not essential for cell growth, survival, and/or productivity. Because expression of these endogenous products consumes considerable cellular energy and DNA/protein building blocks, reducing or eliminating the expression of such endogenous products can render cell growth more efficient and, in the case of cells used to produce a recombinant product of interest, those cellular resources can be diverted to achieve higher productivity of the recombinant product of interest. For example, but not by way of limitation, the further mammalian cell endogenous product selected based on its role in promoting efficient cell growth and higher productivity of a recombinant product of interest is BAX, BAK, ICAM-1, Sirtuin 1 (SIRT-1) or MYC Proto-Oncogene, BHLH Transcription Factor (MYC). In certain embodiments of the subject matter of the herein disclosed inventions, the mammalian cells of the present inventions further exhibit reduced or eliminated expression of SIRT-1. In certain embodiments of the subject matter of the herein disclosed inventions, the mammalian cells of the present inventions further exhibit reduced or eliminated expression of MYC. In certain embodiments of the subject matter of the herein disclosed inventions, the mammalian cells of the present inventions further exhibit reduced or eliminated expression of SIRT-1 and MYC. In certain embodiments of the subject matter of the herein disclosed inventions, the mammalian cells of the present inventions further exhibit reduced or eliminated expression of BAX and MYC. In certain embodiments of the subject matter of the herein disclosed inventions, the mammalian cells of the present inventions further exhibit reduced or eliminated expression of BAK and MYC. In certain embodiments of the subject matter of the herein disclosed inventions, the mammalian cells of the present inventions further exhibit reduced or eliminated expression of ICAM-1 and MYC. In certain embodiments of the subject matter of the herein disclosed inventions, the mammalian cells of the present inventions further exhibit reduced or eliminated expression of BAX and SIRT-1. In certain embodiments of the subject matter of the herein disclosed inventions, the mammalian cells of the present inventions further exhibit reduced or eliminated expression of BAK and SIRT-1. In certain embodiments of the subject matter of the herein disclosed inventions, the mammalian cells of the present inventions further exhibit reduced or eliminated expression of ICAM-1 and SIRT-1. In certain embodiments of the subject matter of the herein disclosed inventions, the mammalian cells of the present inventions further exhibit reduced or eliminated expression of BAX, SIRT-1, and MYC. In certain embodiments of the subject matter of the herein disclosed inventions, the mammalian cells of the present inventions further exhibit reduced or eliminated expression of BAK, SIRT-1, and MYC. In certain embodiments of the subject matter of the herein disclosed inventions, the mammalian cells of the present inventions further exhibit reduced or eliminated expression of ICAM-1, SIRT-1, and MYC. In certain embodiments of the subject matter of the herein disclosed inventions, the mammalian cells of the present inventions further exhibit reduced or eliminated expression of BAX, BAK, SIRT-1, and MYC. In certain embodiments of the subject matter of the herein disclosed inventions, the mammalian cells of the present inventions further exhibit reduced or eliminated expression of BAX, ICAM-1, SIRT-1, and MYC. In certain embodiments of the subject matter of the herein disclosed inventions, the mammalian cells of the present inventions further exhibit reduced or eliminated expression of BAK, ICAM-1, SIRT-1, and MYC. In certain embodiments of the subject matter of the herein disclosed inventions, the mammalian cells of the present inventions further exhibit reduced or eliminated expression of BAX, BAK, MYC, SIRT-1, and ICAM.

In certain embodiments of the subject matter of the herein disclosed inventions, a host cell of the present inventions is modified to reduce or eliminate the expression of one or more host cell endogenous products relative to the expression of the host cell endogenous products in an unmodified, i.e., “reference”, host cell. In certain embodiments of the subject matter of the herein disclosed inventions, the reference host cells are host cells where the expression of one or more particular endogenous product, e.g., a PLOD, especially PLOD1 or/and PLOD2 or/and PLOD3; BAX; BAK; ICAM-1; SIRT-1; and/or MYC polypeptide, is not reduced or eliminated. That is, the reference host cell has the same genotype as the modified cell except for the respective endogenous gene whose expression is reduced or eliminated. In certain embodiments of the subject matter of the herein disclosed inventions, a reference host cell is a cell that comprises at least one or both wild-type alleles of the gene(s) coding for PLOD, especially PLOD1 or/and PLOD2 or/and PLOD3; BAX; BAK; ICAM-1; SIRT-1; and/or MYC. For example, but not by way of limitation, a reference host cell is a host cell that has both wild-type alleles of the gene(s) coding for PLOD, especially PLOD1 or/and PLOD2 or/and PLOD3; BAX; BAK; ICAM-1; SIRT-1; and/or MYC. In certain embodiments of the subject matter of the herein disclosed inventions, the reference host cells are WT host cells. In certain embodiments of the subject matter of the herein disclosed inventions, the modification of reducing or eliminating the expression of one or more host cell endogenous products is performed before the introduction of the exogenous nucleic acid encoding the recombinant product of interest, i.e. the heterologous polypeptide or protein. In certain embodiments of the subject matter of the herein disclosed inventions, the modification of reducing or eliminating the expression of one or more host cell endogenous products is performed after the introduction of the exogenous nucleic acid encoding the recombinant product of interest, i.e. the heterologous polypeptide or protein.

In certain embodiments of the subject matter of the herein disclosed inventions, the expression of one or more endogenous products, e.g., a PLOD, especially PLOD1 or/and PLOD2 or/and PLOD3; BAX; BAK; ICAM-1; SIRT-1; and/or MYC polypeptide, in a cell that has been modified to reduce or eliminate expression of the endogenous product, is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2% or less than about 1% of the corresponding endogenous product expression of a reference cell, e.g., a WT host cell. In certain embodiments of the subject matter of the herein disclosed inventions, the expression of one or more endogenous products in a cell that has been modified to reduce or eliminate expression of the endogenous products, is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2%, or less than about 1% of the corresponding endogenous product expression of a reference cell, e.g., a WT host cell.

In certain embodiments of the subject matter of the herein disclosed inventions, the expression of one or more endogenous products, e.g., a PLOD, especially PLOD1 or/and PLOD2 or/and PLOD3; BAX; BAK; ICAM-1; SIRT-1; and/or MYC polypeptide, in a host cell that has been modified to reduce or eliminate expression of the endogenous products, is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2% or at least about 1% of the corresponding endogenous product expression of a reference host cell, e.g., a WT host cell. In certain embodiments of the subject matter of the herein disclosed inventions, the expression of one or more endogenous products in a host cell that has been modified to reduce or eliminate expression of the endogenous product, is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2%, or at least about 1% of the corresponding endogenous product expression of a reference cell, e.g., a WT mammalian cell.

In certain embodiments of the subject matter of the herein disclosed inventions, the expression of one or more particular endogenous products, e.g., a PLOD, especially PLOD1 or/and PLOD2 or/and PLOD3; BAX; BAK; ICAM-1; SIRT-1; and/or MYC polypeptide, in a cell that has been modified to reduce or eliminate expression of the endogenous products, is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the corresponding endogenous product expression of a reference host cell, e.g., a WT host cell. In certain embodiments of the subject matter of the herein disclosed inventions, the expression of one or more endogenous products, e.g., a PLOD, especially PLOD1 or/and PLOD2 or/and PLOD3; BAX; BAK; ICAM-1; SIRT-1; and/or MYC polypeptide, in a cell that has been modified to reduce or eliminate expression of the endogenous products, is no more than about 40% of the corresponding endogenous product expression of a reference cell, e.g., a WT mammalian cell. In certain embodiments of the subject matter of the herein disclosed inventions, the expression of one or more endogenous products in a cell that has been modified to reduce or eliminate expression of the endogenous products, is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the corresponding endogenous product expression of a reference cell, e.g., a WT host cell.

In certain embodiments of the subject matter of the herein disclosed inventions, the expression of one or more endogenous products, e.g., a PLOD, especially PLOD1 or/and PLOD2 or/and PLOD3; BAX; BAK; ICAM-1; SIRT-1; and/or MYC polypeptide, in a cell that has been modified to reduce or eliminate expression of the endogenous products, is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the corresponding endogenous product expression of a reference cell, e.g., a WT host cell. In certain embodiments of the subject matter of the herein disclosed inventions, the expression of one or more endogenous products, e.g., a PLOD, especially PLOD1 or/and PLOD2 or/and PLOD3; BAX; BAK; ICAM-1; SIRT-1; and/or MYC polypeptide, in a cell that has been modified to reduce or eliminate expression of the endogenous products, is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the corresponding endogenous product expression of a reference cell, e.g., a WT host cell.

In certain embodiments, the expression of one or more endogenous products, e.g., a PLOD, especially PLOD1 or/and PLOD2 or/and PLOD3; BAX; BAK; ICAM-1; SIRT-1; and/or MYC polypeptide, in a cell that has been modified to reduce or eliminate expression of the endogenous products, is between about 5% and about 40% of the corresponding endogenous product expression of a reference cell, e.g., a WT host cell.

In certain embodiments of the subject matter of the herein disclosed inventions, the expression level of the one or more endogenous products, e.g., a PLOD, especially PLOD1 or/and PLOD2 or/and PLOD3; BAX; BAK; ICAM-1; SIRT-1; and/or MYC polypeptide, in different reference cells (e.g., cells that comprise at least one or both wild-type alleles of the corresponding gene) can vary.

In certain embodiments of the subject matter of the herein disclosed inventions, a genetic engineering system is employed to reduce or eliminate the functional expression of one or more particular endogenous products (e.g., PLOD, especially PLOD1 or/and PLOD2 or/and PLOD3; BAX; BAK; ICAM-1; SIRT-1; and/or MYC expression). Various genetic engineering systems known in the art can be used for the methods disclosed herein. Non-limiting examples of such systems include the CRISPR/Cas system, the zinc-finger nuclease (ZFN) system, the transcription activator-like effector nuclease (TALEN) system and the use of other tools for reducing or eliminating protein expression by gene silencing, such as small interfering RNAs (siRNAs), short hairpin RNA (shRNA), and microRNA (miRNA). Any CRISPR/Cas systems known in the art, including traditional, enhanced or modified Cas systems, as well as other bacterial based genome excising tools such as Cpf1 can be used with the methods disclosed herein.

In certain embodiments of the subject matter of the herein disclosed inventions, a portion of one or more genes, e.g., genes coding for a endogenous product such as a PLOD, especially PLOD1 or/and PLOD2 or/and PLOD3; BAX; BAK; ICAM-1; SIRT-1; and/or MYC polypeptide, is deleted to reduce or eliminate expression of the corresponding endogenous product in a host cell. In certain embodiments of the subject matter of the herein disclosed inventions, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85% or at least about 90% of the gene is deleted. In certain embodiments, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85% or no more than about 90% of the gene is deleted. In certain embodiments of the subject matter of the herein disclosed inventions, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of the gene is deleted.

In certain embodiments of the subject matter of the herein disclosed inventions, at least one exon of a gene encoding a PLOD, especially PLOD1 or/and PLOD2 or/and PLOD3; BAX; BAK; ICAM-1; SIRT-1; and/or MYC polypeptide is at least partially deleted in a host cell. The term “partially deleted,” as used herein, refers to at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85%, no more than about 90%, no more than about 95%, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of a region, e.g., of the exon, is deleted.

In certain non-limiting embodiments of the subject matter of the herein disclosed inventions, a CRISPR/Cas9 system is employed to reduce or eliminate the expression of one or more endogenous products, e.g., a PLOD, especially PLOD1 or/and PLOD2 or/and PLOD3; BAX; BAK; ICAM-1; SIRT-1; and/or MYC polypeptide in a host cell. A clustered regularly interspaced short palindromic repeats (CRISPR) system is a genome-editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric) or modular (comprising more than one, and typically two, separate RNA molecules, such as a crRNA and a tracrRNA, which are usually associated with one another, for instance by duplexing).

CRISPR/Cas9 strategies can employ a vector to transfect the mammalian cell. The guide RNA (gRNA) can be designed for each application, as this is the sequence that Cas9 uses to identify and directly bind to the target DNA in a mammalian cell. Multiple crRNAs and the tracrRNA can be packaged together to form a single-guide RNA (sgRNA). The sgRNA can be joined together with the Cas9 gene and made into a vector in order to be transfected into mammalian cells.

In certain embodiments of the subject matter of the herein disclosed inventions, the CRISPR/Cas9 system for use in reducing or eliminating the expression of one or more endogenous products, e.g., a PLOD, especially PLOD1 or/and PLOD2 or/and PLOD3; BAX; BAK; ICAM-1; SIRT-1; and/or MYC polypeptide, comprises a Cas9 molecule and one or more gRNAs comprising a targeting domain that is complementary to a target sequence of the gene encoding the endogenous product or a component thereof. In certain embodiments of the subject matter of the herein disclosed inventions, the target gene is a region of the gene coding for the endogenous product, e.g., a PLOD, especially PLOD1 or/and PLOD2 or/and PLOD3; BAX; BAK; ICAM-1; SIRT-1; and/or MYC polypeptide. The target sequence can be any exon or intron region within the gene.

In certain embodiments of the subject matter of the herein disclosed inventions, the gRNAs are administered to the mammalian cell in a single vector and the Cas9 molecule is administered to the host cell in a second vector. In certain embodiments of the subject matter of the herein disclosed inventions, the gRNAs and the Cas9 molecule are administered to the host cell in a single vector. Alternatively, each of the gRNAs and Cas9 molecules can be administered by separate vectors. In certain embodiments of the subject matter of the herein disclosed inventions, the CRISPR/Cas9 system can be delivered to the host cell as a ribonucleoprotein complex (RNP) that comprises a Cas9 protein complexed with one or more gRNAs, e.g., delivered by electroporation (see, e.g., DeWitt et al., Methods 121-122:9-15 (2017) for additional methods of delivering RNPs to a cell). In certain embodiments of the subject matter of the herein disclosed inventions, administering the CRISPR/Cas9 system to the host cell results in the reduction or elimination of the expression of an endogenous product, e.g., a PLOD, especially PLOD1 or/and PLOD2 or/and PLOD3; BAX; BAK; ICAM-1; SIRT-1; and/or MYC polypeptide.

In certain embodiments of the subject matter of the herein disclosed inventions, the genetic engineering system is a ZFN system for reducing or eliminating the expression of one or more particular endogenous product in a mammalian cell, e.g., a PLOD, especially PLOD1 or/and PLOD2 or/and PLOD3; BAX; BAK; ICAM-1; SIRT-1; and/or MYC polypeptide. The ZFN can act as a restriction enzyme, which is generated by combining a zinc finger DNA-binding domain with a DNA-cleavage domain. A zinc finger domain can be engineered to target specific DNA sequences, which allows the zinc-finger nuclease to target desired sequences within genomes. The DNA-binding domains of individual ZFNs typically contain a plurality of individual zinc finger repeats and can each recognize a plurality of base pairs. The most common method to generate a new zinc-finger domain is to combine smaller zinc-finger “modules” of known specificity. The most common cleavage domain in ZFNs is the non-specific cleavage domain from the type II restriction endonuclease FokI. ZFN modulates the expression of proteins by producing double-strand breaks (DSBs) in the target DNA sequence, which will, in the absence of a homologous template, be repaired by non-homologous end-joining (NHEJ). Such repair can result in deletion or insertion of base pairs, producing frame-shift and preventing the production of the harmful protein (Durai et al., Nucleic Acids Res.; 33 (18): 5978-90 (2005)). Multiple pairs of ZFNs can also be used to completely remove entire large segments of genomic sequence (Lee et al., Genome Res.; 20 (1): 81-9 (2010)).

In certain embodiments of the subject matter of the herein disclosed inventions, the genetic engineering system is a TALEN system for reducing or eliminating the expression of one or more particular endogenous product, e.g., a PLOD, especially PLOD1 or/and PLOD2 or/and PLOD3; BAX; BAK; ICAM-1; SIRT-1; and/or MYC polypeptide, in a mammalian cell. TALENs are restriction enzymes that can be engineered to cut specific sequences of DNA. TALEN systems operate on a similar principle as ZFNs. TALENs are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et al., Nature Biotechnology; 29 (2): 135-6 (2011)). In certain embodiments of the subject matter of the herein disclosed inventions, the target gene encodes a PLOD, especially PLOD1 or/and PLOD2 or/and PLOD3; BAX; BAK; ICAM-1; SIRT-1; and/or MYC polypeptide.

In certain embodiments of the subject matter of the herein disclosed inventions, the expression of one or more particular endogenous product, e.g., a PLOD, especially PLOD1 or/and PLOD2 or/and PLOD3; BAX; BAK; ICAM-1; SIRT-1; and/or MYC polypeptide, can be reduced or eliminated using oligonucleotides that have complementary sequences to corresponding nucleic acids (e.g., mRNA). Non-limiting examples of such oligonucleotides include small interference RNA (siRNA), short hairpin RNA (shRNA), and micro RNA (miRNA). In certain embodiments of the subject matter of the herein disclosed inventions, such oligonucleotides can be homologous to at least a portion of a PLOD, especially PLOD1 or/and PLOD2 or/and PLOD3; BAX; BAK; ICAM-1; SIRT-1; and/or MYC polypeptide nucleic acid sequence, wherein the homology of the portion relative to the corresponding nucleic acid sequence is at least about 75 or at least about 80 or at least about 85 or at least about 90 or at least about 95 or at least about 98 percent. In certain non-limiting embodiments of the subject matter of the herein disclosed inventions, the complementary portion can constitute at least 10 nucleotides or at least 15 nucleotides or at least 20 nucleotides or at least 25 nucleotides or at least 30 nucleotides and the antisense nucleic acid, shRNA, mRNA or siRNA molecules can be up to 15 or up to 20 or up to 25 or up to 30 or up to 35 or up to 40 or up to 45 or up to 50 or up to 75 or up to 100 nucleotides in length. Antisense nucleic acid, shRNA, mRNA or siRNA molecules can comprise DNA or atypical or non-naturally occurring residues, for example, but not limited to, phosphorothioate residues.

The genetic engineering systems disclosed herein can be delivered into the mammalian cell using a viral vector, e.g., retroviral vectors such as gamma retroviral vectors, and lentiviral vectors. Combinations of retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify the mammalian cells disclosed herein. In certain embodiments of the subject matter of the herein disclosed inventions, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic engineering of the mammalian cell disclosed herein. For example, a nucleic acid molecule can be introduced into the mammalian cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation and protoplast fusion. Liposomes can also be potentially beneficial for delivery of nucleic acid molecules into a mammalian cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

### Targeted Integration

Targeted integration (TI) allows exogenous nucleotide sequences to be integrated into a pre-determined site of a mammalian cell's genome.

In certain embodiments of the subject matter of the herein disclosed inventions, the use of a TI host cell for the introduction of an exogenous nucleic acid encoding a heterologous polypeptide or protein of interest will provide for robust, stable cell culture performance and lower risk of unwanted secondary modifications in the resulting recombinant product of interest. TI host cells and strategies for the use of the same are described in detail in US 2021/0002669, the contents of which are incorporated by reference in their entirety.

In certain embodiments of the subject matter of the herein disclosed inventions employing targeted integration, the exogenous nucleotide sequence encoding the heterologous polypeptide or protein is integrated at a site within a specific locus of the genome of a TI host cell. In certain embodiments of the subject matter of the herein disclosed inventions, the locus into which the exogenous nucleotide sequence encoding the heterologous polypeptide or protein is integrated is at least about 50%, at least about 60%, at least about 70%, at least about 80%, at least about 90%, at least about 95%, at least about 99%, or at least about 99.9% homologous to a sequence selected from Contigs NW_006874047.1, NW_006884592.1, NW 006881296.1, NW 003616412.1, NW_003615063.1, NW_006882936.1, and NW_003615411.1.

In certain embodiments of the subject matter of the herein disclosed inventions, the nucleotide sequence (immediately) 5′ of the integrated exogenous sequence encoding the heterologous polypeptide or protein is selected from the group consisting of nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW 003615063.1, nucleotides 2650443-2662054 of NW_006882936.1, or nucleotides 82214-97705 of NW_003615411.1 and sequences at least 50% homologous thereto. In certain embodiments of the subject matter of the herein disclosed inventions, the nucleotide sequence (immediately) 5′ of the integrated exogenous sequence encoding the heterologous polypeptide or protein are at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW 006884592.1, nucleotides 253831-491909 of NW 006881296.1, nucleotides 69303-79768 of NW_003616412.1, nucleotides 293481-315265 of NW 003615063.1, nucleotides 2650443-2662054 of NW 006882936.1, or nucleotides 82214-97705 of NW 003615411.1.

In certain embodiments of the subject matter of the herein disclosed inventions, the nucleotide sequence (immediately) 3′ of the integrated exogenous sequence encoding the heterologous polypeptide or protein is selected from the group consisting of nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1 and sequences at least 50% homologous thereto. In certain embodiments of the subject matter of the herein disclosed inventions, the nucleotide sequence (immediately) 3′ of the integrated exogenous sequence encoding the heterologous polypeptide or protein is at least about 50%, at least about 60%, at least about 70%, at least about 80%, at least about 90%, at least about 95%, at least about 99%, or at least about 99.9% homologous to nucleotides 45270-45490 of NW_006874047.1, nucleotides 207912-792374 of NW 006884592.1, nucleotides 491910-667813 of NW 006881296.1, nucleotides 79769-100059 of NW 003616412.1, nucleotides 315266-362442 of NW_003615063.1, nucleotides 2662055-2701768 of NW_006882936.1, or nucleotides 97706-105117 of NW 003615411.1.

In certain embodiments of the subject matter of the herein disclosed inventions, the integrated exogenous sequence encoding the heterologous polypeptide or protein is flanked 5′ by a nucleotide sequence selected from the group consisting of nucleotides 41190-45269 of NW_006874047.1, nucleotides 63590-207911 of NW_006884592.1, nucleotides 253831-491909 of NW 006881296.1, nucleotides 69303-79768 of NW_003616412.1, nucleotides 293481-315265 of NW 003615063.1, nucleotides 2650443-2662054 of NW_006882936.1, and nucleotides 82214-97705 of NW_003615411.1, and sequences at least 50% homologous thereto. In certain embodiments of the subject matter of the herein disclosed inventions, the integrated exogenous sequence encoding the heterologous polypeptide or protein is flanked 3′ by a nucleotide sequence selected from the group consisting of nucleotides 45270-45490 of NW_006874047.1, nucleotides 207912-792374 of NW_006884592.1, nucleotides 491910-667813 of NW 006881296.1, nucleotides 79769-100059 of NW_003616412.1, nucleotides 315266-362442 of NW 003615063.1, nucleotides 2662055-2701768 of NW_006882936.1, and nucleotides 97706-105117 of NW_003615411.1 and sequences at least 50% homologous thereto. In certain embodiments of the subject matter of the herein disclosed inventions, the nucleotide sequence flanking 5′ of the integrated exogenous nucleotide sequence encoding the heterologous polypeptide or protein is at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW 006884592.1, nucleotides 253831-491909 of NW 006881296.1, nucleotides 69303-79768 of NW_003616412.1, nucleotides 293481-315265 of NW_003615063.1, nucleotides 2650443-2662054 of NW_006882936.1, and nucleotides 82214-97705 of NW_003615411.1. In certain embodiments of the subject matter of the herein disclosed inventions, the nucleotide sequence flanking 3′ of the integrated exogenous nucleotide sequence encoding the heterologous polypeptide or protein is at least about 50%, at least about 60%, at least about 70%, at least about 80%, at least about 90%, at least about 95%, at least about 99%, or at least about 99.9% homologous to nucleotides 45270-45490 of NW 006874047.1, nucleotides 207912-792374 of NW_006884592.1, nucleotides 491910-667813 of NW 006881296.1, nucleotides 79769-100059 of NW_003616412.1, nucleotides 315266-362442 of NW 003615063.1, nucleotides 2662055-2701768 of NW_006882936.1, and nucleotides 97706-105117 of NW_003615411.1.

In certain embodiments of the subject matter of the herein disclosed inventions, the integrated exogenous nucleotide sequence encoding the heterologous polypeptide or protein is operably linked to a nucleotide sequence selected from the group consisting of Contigs NW_006874047.1, NW_006884592.1, NW_006881296.1, NW_003616412.1, NW 003615063.1, NW_006882936.1, and NW_003615411.1 and sequences at least 50% homologous thereto. In certain embodiments of the subject matter of the herein disclosed inventions, the nucleotide sequence operably linked to the exogenous nucleotide sequence encoding the heterologous polypeptide or protein is at least about 50%, at least about 60%, at least about 70%, at least about 80%, at least about 90%, at least about 95%, at least about 99%, or at least about 99.9% homologous to a sequence selected from Contigs NW_006874047.1, NW_006884592.1, NW_006881296.1, NW_003616412.1, NW_003615063.1, NW 006882936.1, and NW_003615411.1.

In targeted integration, site-specific recombination is employed for the introduction of an exogenous nucleic acid into a specific locus in the genome of a mammalian TI host cell. This is an enzymatic process wherein a sequence at the site of integration in the genome is exchanged for the exogenous nucleic acid. One system used to effect such nucleic acid exchanges is the Cre-lox system. The enzyme catalyzing the exchange is the Cre recombinase. The sequence to be exchanged is defined by the position of two lox (P)-sites in the genome as well as in the exogenous nucleic acid. These lox (P)-sites are recognized by the Cre recombinase. Nothing more is required, i.e., no ATP etc. Originally, the Cre-lox system has been found in bacteriophage P1.

In certain embodiments of all aspects and embodiments of the subject matter of the herein disclosed inventions, the cell modified according to the subject matter of the current inventions has been prior to the modification according to the subject matter of the current disclosure subjected to targeted integration of nucleic acid(s) encoding a heterologous polypeptide or protein of interest.

In certain embodiments of all aspects and embodiments of the subject matter of the current inventions, the cell modified according to the subject matter of the current inventions has been after the modification according to the subject matter of the current inventions subjected to targeted integration of nucleic acid(s) encoding a heterologous polypeptide or protein of interest.

In certain embodiments of all aspects and embodiments of the subject matter of the current inventions, the targeted integration is mediated by a recombinase that recognizes one or more recombination recognition sequences (RRSs), which are present in the genome of the mammalian cell and in the exogenous nucleotide sequence encoding the heterologous polypeptide or protein to be integrated into the genome of the mammalian cell.

In certain embodiments of all aspects and embodiments of the subject matter of the current inventions, the targeted integration is mediated by homologous recombination.

A “recombination recognition sequence” (RRS) is a nucleotide sequence recognized by a recombinase and is necessary and sufficient for recombinase-mediated recombination events. A RRS can be used to define the position where a recombination event will occur in a nucleotide sequence.

In certain embodiments of all aspects and embodiments of the subject matter of the current inventions, the RRS is recognized by a Cre recombinase.

In certain embodiments of all aspects and embodiments of the subject matter of the current inventions, the RRS is a LoxP site and a Cre recombinase mediates the targeted integration by recombination.

In certain embodiments of all aspects and embodiments of the subject matter of the current inventions, the RRS is recognized by a FLP recombinase.

In certain embodiments of all aspects and embodiments of the subject matter of the current inventions, the RRS is a FRT site and a FLP recombinase mediates the targeted integration by recombination.

In certain embodiments of all aspects and embodiments of the subject matter of the current inventions, the RRS is recognized by a Bxb1 integrase.

In certain embodiments of all aspects and embodiments of the subject matter of the current inventions, the RRS is a Bxb1 attP or a Bxb1 attB site and a Bxb1 integrase mediates the targeted integration by recombination.

In certain embodiments of all aspects and embodiments of the subject matter of the current inventions, the RRS is recognized by a C31 integrase.

In certain embodiments of all aspects and embodiments of the subject matter of the current inventions, the RRS is a QC31 attP or a QC31 attB site and the PC31 integrase mediates the targeted integration by recombination.

The recombinases can be introduced into a cell using an expression vector comprising coding sequences of the enzymes or as protein or an mRNA.

With respect to TI, any known or future mammalian cell including those modified according to the subject matter of the current inventions comprising a landing site as described herein integrated at a single site within a locus of the genome can be used in the subject matter of the current inventions. Such a cell is denoted as a mammalian TI host cell.

In certain embodiments of all aspects and embodiments of the subject matter of the current inventions, the mammalian TI host cell is a hamster cell, a human cell, a rat cell, or a mouse cell comprising a landing site as described herein. In one preferred embodiment of the subject matter of the herein disclosed inventions, the mammalian TI host cell is a CHO cell. In certain embodiments of the subject matter of the herein disclosed inventions, the mammalian TI CHO host cell is a CHO K1 cell, a CHO KISV cell, a CHO DG44 cell, a CHO DUKXB-11 cell, a CHO KIS cell, or a CHO KIM cell comprising a landing site as described herein integrated at a single site within a locus of the genome.

In certain embodiments of all aspects and embodiments of the subject matter of the current inventions, a mammalian TI host cell comprises an integrated landing site, wherein the landing site comprises one or more recombination recognition sequence (RRS). The RRS can be recognized by a recombinase, for example, a Cre recombinase, an FLP recombinase, a Bxb1 integrase, or a QC31 integrase. The one or more RRS can be selected independently of each other from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a QC31 attP sequence, and a QC31 attB sequence. If multiple RRSs have to be present, the selection of each of the sequences is dependent on the other insofar as non-identical RRSs are chosen.

In certain embodiments of all aspects and embodiments of the subject matter of the current inventions, the landing site comprises one or more recombination recognition sequence (RRS), wherein the RRS can be recognized by a recombinase. In certain embodiments of the subject matter of the herein disclosed inventions, the integrated landing site comprises at least two RRSs. In certain embodiments of the subject matter of the herein disclosed inventions, an integrated landing site comprises three RRSs, wherein the third RRS is located between the first and the second RRS. In certain preferred embodiments of the subject matter of the herein disclosed inventions, all three RRSs are different. In certain embodiments of the subject matter of the herein disclosed inventions, the landing site comprises a first, a second and a third RRS, and at least one selection marker located between the first and the second RRS, and the third RRS is different from the first and/or the second RRS. In certain embodiments of the subject matter of the herein disclosed inventions, the landing site further comprises a second selection marker, and the first and the second selection markers are different. In certain embodiments of the subject matter of the herein disclosed inventions, the landing site further comprises a third selection marker and an internal ribosome entry site (IRES), wherein the IRES is operably linked to the third selection marker. The third selection marker can be different from the first or the second selection marker.

An exemplary mammalian TI host cell that is suitable for modification according to the subject matter of the current inventions as well as for use in a method according to the subject matter of the current inventions is a CHO cell harboring a landing site integrated at a single site within a locus of its genome wherein the landing site comprises three heterospecific loxP sites for Cre recombinase mediated DNA recombination.

In this example, the heterospecific loxP sites are L3, LoxFas and 2L (see e.g., Lanza et al., Biotechnol. J. 7 (2012) 898-908; Wong et al., Nucleic Acids Res. 33 (2005) e147), whereby L3 and 2L flank the landing site at the 5′-end and 3′-end, respectively, and LoxFas is located between the L3 and 2L sites. The landing site further contains a bicistronic unit linking the expression of a selection marker via an IRES to the expression of the fluorescent GFP protein allowing to stabilize the landing site by positive selection as well as to select for the absence of the site after transfection and Cre-recombination (negative selection). Green fluorescence protein (GFP) serves for monitoring the RMCE reaction.

Such a configuration of the landing site as outlined in the previous paragraph allows for the simultaneous integration of two vectors, e.g., of a so called front vector harboring an L3 and a LoxFas site and a back vector harboring a LoxFas and an 2L site. The functional elements of a selection marker gene different from that present in the landing site can be distributed between both vectors: promoter and start codon can be located on the front vector whereas coding region and poly A signal are located on the back vector. Only correct recombinase-mediated integration of said nucleic acids from both vectors induces resistance against the respective selection agent.

Generally, a mammalian TI host cell is a mammalian cell comprising a landing site integrated at a single site within a locus of the genome of the mammalian cell, wherein the landing site comprises a first and a second recombination recognition sequence flanking at least one first selection marker, and a third recombination recognition sequence located between the first and the second recombination recognition sequence, and all the recombination recognition sequences are different.

The selection marker(s) can be selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. The selection marker(s) can also be a fluorescent protein selected from the group consisting of green fluorescent protein (GFP), enhanced GFP (eGFP), a synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald6, CyPet, mCFPm, Cerulean, and T-Sapphire.

An exogenous nucleotide sequence is a nucleotide sequence that does not originate from a specific cell but can be introduced into said cell by DNA delivery methods, such as, e.g., by transfection, electroporation, or transformation methods. In certain embodiments of the subject matter of the herein disclosed inventions, a mammalian TI host cell comprises at least one landing site integrated at one or more integration sites in the mammalian cell's genome. In certain embodiments of the subject matter of the herein disclosed inventions, the landing site is integrated at one or more integration sites within a specific locus of the genome of the mammalian cell.

In certain embodiments of all aspects and embodiments according to the subject matter of the current inventions, the integrated landing site comprises at least one selection marker. In certain embodiments of the subject matter of the herein disclosed inventions, the integrated landing site comprises a first, a second and a third RRS, and at least one selection marker. In certain embodiments, a selection marker is located between the first and the second RRS. In certain embodiments of the subject matter of the herein disclosed inventions, two RRSs flank at least one selection marker, i.e., a first RRS is located 5′ (upstream) and a second RRS is located 3′ (downstream) of the selection marker. In certain embodiments of the subject matter of the herein disclosed inventions, a first RRS is adjacent to the 5′-end of the selection marker and a second RRS is adjacent to the 3′-end of the selection marker. In certain embodiments, the landing site comprises a first, second, and third RRS, and at least one selection marker located between the first and the third RRS.

In certain embodiments of all aspects and embodiments according to the subject matter of the current inventions, a selection marker is located between a first and a second RRS and the two flanking RRSs are different. In certain preferred embodiments of the subject matter of the herein disclosed inventions, the first flanking RRS is a LoxP L3 sequence and the second flanking RRS is a LoxP 2L sequence. In certain embodiments of the subject matter of the herein disclosed inventions, a LoxP L3 sequence is located 5′ of the selection marker and a LoxP 2L sequence is located 3′ of the selection marker. In certain embodiments of the subject matter of the herein disclosed inventions, the first flanking RRS is a wild-type FRT sequence and the second flanking RRS is a mutant FRT sequence. In certain embodiments of the subject matter of the herein disclosed inventions, the first flanking RRS is a Bxb 1 attP sequence and the second flanking RRS is a Bxb1 attB sequence.

In certain embodiments of the subject matter of the herein disclosed inventions, the first flanking RRS is a QC31 attP sequence and the second flanking RRS is a QC31 attB sequence. In certain embodiments of the subject matter of the herein disclosed inventions, the two RRSs are positioned in the same orientation. In certain embodiments of the subject matter of the herein disclosed inventions, the two RRSs are both in the forward or reverse orientation. In certain embodiments of the subject matter of the herein disclosed inventions, the two RRSs are positioned in opposite orientations.

In certain embodiments of all aspects and embodiments according to the subject matter of the current inventions, the integrated landing site comprises a first and a second selection marker, which are flanked by two RRSs, wherein the first selection marker is different from the second selection marker. In certain embodiments of the subject matter of the herein disclosed inventions, the two selection markers are both independently of each other selected from the group consisting of a glutamine synthetase selection marker, a thymidine kinase selection marker, a HYG selection marker, and a puromycin resistance selection marker. In certain embodiments of the subject matter of the herein disclosed inventions, the integrated landing site comprises a thymidine kinase selection marker and a HYG selection marker. In certain embodiments of the subject matter of the herein disclosed inventions, the first selection maker is selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid, and the second selection maker is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire fluorescent protein. In certain embodiments of the subject matter of the herein disclosed inventions, the first selection marker is a glutamine synthetase selection marker and the second selection marker is a GFP fluorescent protein. In certain embodiments of the subject matter of the herein disclosed inventions, the two RRSs flanking both selection markers are different.

In certain embodiments of the subject matter of the herein disclosed inventions, the selection marker is operably linked to a promoter sequence. In certain embodiments of the subject matter of the herein disclosed inventions, the selection marker is operably linked to an SV40 promoter. In certain embodiments of the subject matter of the herein disclosed inventions, the selection marker is operably linked to a human Cytomegalovirus (CMV) promoter.

The Cre-lox system operates in different cell types, like mammals, plants, bacteria and yeast.

In certain embodiments of all aspects and embodiments according to the subject matter of the current inventions, the exogenous nucleic acid encoding the heterologous polypeptide or protein has been integrated into the mammalian TI host cell by single or double recombinase mediated cassette exchange (RMCE). Thereby a recombinant mammalian cell, such as a recombinant CHO cell, is obtained, in which a defined and specific expression cassette sequence has been integrated into the genome at a single locus, which in turn results in the efficient expression and production of the heterologous polypeptide.

The Cre-LoxP site-specific recombination system has been widely used in many biological experimental systems. Cre recombinase is a 38-kDa site-specific DNA recombinase that recognizes 34 bp LoxP sequences. Cre recombinase is derived from bacteriophage P1 and belongs to the tyrosine family site-specific recombinase. Cre recombinase can mediate both intra- and intermolecular recombination between LoxP sequences. The LoxP sequence is composed of an 8 bp non-palindromic core region flanked by two 13 bp inverted repeats. Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre recombinase-mediated recombination will excise DNA sequences located between the two LoxP sequences as a covalently closed circle. If two LoxP sequences are placed in an inverted position on the same nucleotide sequence, Cre recombinase-mediated recombination will invert the orientation of the DNA sequences located between the two sequences. If two LoxP sequences are on two different DNA molecules and if one DNA molecule is circular, Cre recombinase-mediated recombination will result in integration of the circular DNA sequence.

The term “matching RRSs” indicates that a recombination occurs between two RRSs. In certain embodiments of the subject matter of the herein disclosed inventions, the two matching RRSs are the same. In certain embodiments of the subject matter of the herein disclosed inventions, both RRSs are wild-type LoxP sequences. In certain embodiments of the subject matter of the herein disclosed inventions, both RRSs are mutant LoxP sequences. In certain embodiments of the subject matter of the herein disclosed inventions, both RRSs are wild-type FRT sequences. In certain embodiments of the subject matter of the herein disclosed inventions, both RRSs are mutant FRT sequences. In certain embodiments of the subject matter of the herein disclosed inventions, the two matching RRSs are different sequences but can be recognized by the same recombinase. In certain embodiments of the subject matter of the herein disclosed inventions, the first matching RRS is a Bxb1 attP sequence and the second matching RRS is a Bxb1 attB sequence. In certain embodiments of the subject matter of the herein disclosed inventions, the first matching RRS is a QC31 attB sequence and the second matching RRS is a QC31 attB sequence.

A “two-plasmid RMCE” strategy or “double RMCE” is employed in the method according to the subject matter of the current inventions when using a two-vector combination. For example, but not by way of limitation, an integrated landing site could comprise three RRSs, e.g., an arrangement where the third RRS (“RRS3”) is present between the first RRS (“RRS1”) and the second RRS (“RRS2”), while a first vector comprises two RRSs matching the first and the third RRS on the integrated exogenous nucleotide sequence, and a second vector comprises two RRSs matching the third and the second RRS on the integrated exogenous nucleotide sequence.

The two-plasmid RMCE strategy involves using three RRS sites to carry out two independent RMCEs simultaneously. Therefore, a landing site in the mammalian TI host cell using the two-plasmid RMCE strategy includes a third RRS site (RRS3) that has no cross activity with either the first RRS site (RRS1) or the second RRS site (RRS2). The two plasmids to be targeted require the same flanking RRS sites for efficient targeting, one plasmid (front) flanked by RRS1 and RRS3 and the other (back) by RRS3 and RRS2. In addition, two selection markers are needed in the two-plasmid RMCE. One selection marker expression cassette was split into two parts. The front plasmid would contain the promoter followed by a start codon and the RRS3 sequence. The back plasmid would have the RRS3 sequence fused to the N-terminus of the selection marker coding region, minus the start-codon (ATG). Additional nucleotides may need to be inserted between the RRS3 site and the selection marker sequence to ensure in-frame translation for the fusion protein, i.e., operable linkage. Only when both plasmids are correctly inserted, the full expression cassette of the selection marker will be assembled and, thus, rendering cells resistant to the respective selection agent.

Two-plasmid RMCE involves double recombination crossover events, catalyzed by a recombinase, between the two heterospecific RRSs within the target genomic locus and the donor DNA molecule. Two-plasmid RMCE is designed to introduce a copy of the DNA sequences from the front- and back-vector in combination into the pre-determined locus of a mammalian TI host cell's genome. RMCE can be implemented such that prokaryotic vector sequences are not introduced into the mammalian TI host cell's genome, thus, reducing and/or preventing unwanted triggering of host immune or defense mechanisms. The RMCE procedure can be repeated with multiple DNA sequences.

In certain embodiments of all aspects and embodiments according to the subject matter of the current inventions, targeted integration is achieved by two RMCEs, wherein two different DNA sequences, each comprising at least one expression cassette encoding a part of a heteromultimeric polypeptide and/or at least one selection marker or part thereof flanked by two heterospecific RRSs, are both integrated into a pre-determined site of the genome of a RRSs matching mammalian TI host cell. In certain embodiments of the subject matter of the herein disclosed inventions, targeted integration is achieved by multiple RMCEs, wherein DNA sequences from multiple vectors, each comprising at least one expression cassette encoding a part of a heteromultimeric polypeptide and/or at least one selection marker or part thereof flanked by two heterospecific RRSs, are all integrated into a predetermined site of the genome of a mammalian TI host cell. In certain embodiments of the subject matter of the herein disclosed inventions, the selection marker can be partially encoded on the first the vector and partially encoded on the second vector such that only the correct integration of both by double RMCE allows for the expression of the selection marker.

It has to be pointed out that, in certain embodiments of the subject matter of the herein disclosed inventions, the reduction of the transcriptional activity, e.g., by knock-out, can be performed either before introduction of the exogenous nucleic acid encoding the heterologous polypeptide or protein and likewise also thereafter in a stable transfected clone or a stable transfected pool of clones.

### Specific Embodiments of the Compositions and Methods According to the Current Inventions

The presence of hydroxylated lysine residues in a recombinantly produced heterologous product protein requires laborious and time consuming assessments and monitoring for biological activity, pharmacokinetic, immunogenicity and safety.

It has been found that in recombinantly produced antibodies the modification of lysine to hydroxy lysine occurs at different lysine residues with different extent, i.e. also at lysine residues that were not part of the Xaa-Lys-Gly consensus sequence.

This has been shown for three trivalent bispecific antibodies (TCB format) with different binding specificities. The respective results are shown in the following Table.

| | | | | |
|---|---|---|---|---|
| | | | modified | hydroxy lysine |
| | | | amino | content |
| | | | acid | determined |
| identifier | sequence (determined via LC-MS) | location | residue | by LC-MS [%] |

antibody 1

| | | | | |
|---|---|---|---|---|
| LC (1) | ***********VKG* | CDR-2 | K | 0.0254 |
| LC (1′) | ************VK(G) | CDR-2 | K | 0.0044 |
| HC (1) | LTVLSSASTK(G) (SEQ ID NO: 54) | variable/CH1 | K | 4.9179 |
| HC (2) | LTVLSSASTKGPSVFPLAPSSK(S) (SEQ ID NO: 55) | variable/CH1 | K | 7.4339 |

antibody 2

| | | | | |
|---|---|---|---|---|
| LC (1) | ***********VKG* | CDR-2 | K | 0.0201 |
| HC (3) | *************VTVSSASTK(G) (SEQ ID NO: 56) | variable/CH1 | K | 0.5275 |
| HC (4) | *************VTVSSASTKGPSVFPLAPSSK(S) | variable/CH1 | K | 0.0493 |
| | (SEQ ID NO: 57) | | | |
| LC (2) | LKSGTASVVCLLNNFYPR (SEQ ID NO: 61) | CL | K | 0.0052 |
| LC (3) | DSTYSLSSTLTLSKADYEK(H) (SEQ ID NO: 62) | CL | K | 0.0030 |
| LC (3′) | DSTYSLSSTLTLSK(A) (SEQ ID NO: 63) | CL | K | 0.0097 |
| HC (5) | ****SSASTK(G) (SEQ ID NO: 58) | variable/CH1 | K | 1.1074 |
| HC (6) | ****SSASTKGPSVFPLAPSSK(S) (SEQ ID NO: 59) | variable/CH1 | K | 1.1500 |

antibody 3

| | | | | |
|---|---|---|---|---|
| LC (1) | ***********VKG* | CDR-2 | K | 0.0312 |
| LC (1′) | ***********VK(G) | CDR-2 | K | 0.0967 |
| HC-H | NQVSLSCAVK(G) (SEQ ID NO: 60) | CH3 | K | 0.0789 |
| HC-K (1) | ****SSASTK(G) (SEQ ID NO: 64) | variable/CH1 | K | 3.5692 |
| HC-K (2) | ****SSASTKGPSVFPLAPSSK(S) (SEQ ID NO: 65) | variable/CH1 | K | 3.1741 |

It has now been found that the formation of hydroxylysins are based on the action of procollagen-lysine,2-oxoglutarate 5-dioxygenase.

The term “procollagen-lysine,2-oxoglutarate 5-dioxygenase” denotes a protein with enzymatic activity, which is encoded by the nucleic acid sequence of the PLOD gene, or a nucleic acid 5 sequence homologous to it. Said term encompasses also proteins derived from the complete mRNA sequence of PLOD or a splice variant thereof, such as, e.g., for PLOD2 LH2a and LH2b. The inventions also encompass knockout of PLOD2 variants, which are, e.g., differing in nucleotide sequence or even in amino acid sequence of the encoded protein as long as the protein catalyzes the hydroxylation of lysine residues. As a result of the degeneracy of the genetic code, a multitude of different polynucleotide sequences can encode “procollagen-10 lysine,2-oxoglutarate 5-dioxygenase 2”. However, in the genome only a single one thereof is present.

PLODs are located in the rough ER. These enzymes are responsible for lysine hydroxylation. These enzymes require as cofactors Fe2, ascorbate and α-ketoglutarate (aKG). The oxidative decomposition of aKG forms CO2 plus succinate and leads to the generation of an Fe(IV)-oxo or other activated oxygen species that hydroxylate the primary substrate. A complex composed of PLOD1, P3H3 and P3H4 that catalyzes hydroxylation of lysine residues in collagen alpha chains and is required for normal assembly and cross-linking of collagen fibrils.

PLOD2s have been reported, e.g., by Ruotsalainen, H., et al. (Matrix Biol. 18 (1999) 325-329 and 20 (2001) 137-146) and Valtavaara, M., et al. (J. Biol. Chem. 272 (1997) 6831-6834). Yeowell and Walker especially have reported the presence of splice variants (Matrix Biol. 18 (1999) 179-187).

Three genes encoding a “procollagen-lysine,2-oxoglutarate 5-dioxygenase” (PLOD) have been identified: PLOD1, PLOD2 and PLOD3. The proteins show enzymatic activity towards the synthetic peptides ARGIKGIRGFS (SEQ ID NO: 51), GIKGIKGIKGIK (SEQ ID NO: 52), and IKGIKGIKG (SEQ ID NO: 53) (Scietti, L., et al., Nat. Comm. 9 (2018) 3136). The detailed substrate specificity is not known. See further, Valtavaara, M., et al., J. Biol. Chem. 272 (1997) 6831-6834 and 273 (1998) 12881-12886; Passoja, K., et al., Proc. Natl. Acad. Sci. USA 95 (1998) 10482-10486; and Scietti, L., et al., Nat. Comm. 9 (2018) 3136.

The PLOD1 and PLOD3 genes comprise 19 exons, wherein the intron/exon boundaries are identical. The PLOD2 gene is highly homologous to PLOD1 and PLOD3. However, it comprises in comparison to PLOD1 and PLOD3 an additional exon between exon 13 and exon 14, which is denoted as exon 13A. For PLOD2 two splice variant can be expressed (LH2a and LH2b), where LH2b differs from LH2a by incorporating exon 13A.

The subject matter of the herein disclosed inventions is based at least in part on the finding that the knockout of the PLOD gene reduces the level of/abolish the formation of hydroxy lysine in recombinantly produced heterologous proteins.

The subject matter of the herein disclosed inventions is based at least in part on the finding that the knockout of the PLOD gene does not affect overall cell culture performance of the respective modified cell. It has been found that PLOD knockout cell lines have higher biomass formation capacity during cultivation and thereby higher recombinant protein titer compared to a cell of the identical genotype except for the PLOD knockout.

The subject matter of the herein disclosed inventions is based at least in part on the finding that the knockout of the PLOD gene does not affect other protein quality attributes. It has been found that CE-SDS and SEC assessment shows comparable or even increased main peak levels and comparable or even reduced side product levels.

The subject matter of the herein disclosed inventions is based at least in part on the finding that the knockout of the PLOD gene reduces filterability issues. Without being bound by this theory, it is assumed that this is due to the fact that collagen cannot be cross-linked any more, especially as CHO cells express many collagen genes in high levels. It has been found that filterability was at least comparable or even improved by reduced filter pressure increase.

The subject matter of the herein disclosed inventions is based at least in part on the finding that the knockout of the PLOD gene reduces cell aggregation during cultivation. Without being bound by this theory, it is assumed that due to the reduction/absence of cross-linked collagen trans binding of different CHO cells via integrin α1β1 cannot be mediated.

The deletion of the PLOD enzymatic activity can be effected by specific gene product(s) inactivation, e.g. via CRISPR/Cas-based gene knockout, zinc finger nucleases-based knockout etc. The inactivation/deletion of enzymatic activity introduced into a host cell allows for subsequent use in different projects. However, off-target effects and putatively interfering with other important cell performance (e.g. cell growth and productivity) or quality attributes were unexpectedly not found.

This has been shown for the same three trivalent bispecific antibodies with different binding specificities the formation of hydroxylated lysine can be reduced by applying the method according to the current inventions. At the same time the obtained titer has been increased. The respective results are shown in the following Tables.

| | | | | | | |
|---|---|---|---|---|---|---|
| | | | | hydroxy | hydroxy | |
| | | | | lysine | lysine | |
| | | | | content | content | |
| | | | | prior to | after | |
| | | | | PLOD1-3 | PLOD1-3 | |
| | | | modified | knock-out | knock-out | |
| | | | amino | determined | determined | |
| | | | acid | by LC-MS | by LC-MS | reduction |
| identifier | sequence (determined via LC-MS) | location | residue | [%] | [%] | by [%] |

antibody 1

| | | | | | | |
|---|---|---|---|---|---|---|
| LC (1) | ***********VKG* | CDR-2 | K | 0.0254 | 0.0053 | 79 |
| LC (1′) | ************VK(G) | CDR-2 | K | 0.0044 | 0.0004 | 91 |
| HC (1) | LTVLSSASTK(G) | variable/CH1 | K | 4.9179 | 0.1608 | 97 |
| HC (2) | LTVLSSASTKGPSVFPLAPSSK(S) | variable/CH1 | K | 7.4339 | 0.7108 | 90 |

antibody 2

| | | | | | | |
|---|---|---|---|---|---|---|
| LC (1) | ***********VKG* | CDR-2 | K | 0.0201 | 0.0070 | 65 |
| HC (3) | *************VTVSSASTK(G) | variable/CH1 | K | 0.5275 | 0.0387 | 93 |
| HC (4) | *************VTVSSASTKGPSVFPLAPSSK(S) | variable/CH1 | K | 0.0493 | 0.0081 | 84 |
| LC (2) | LKSGTASVVCLLNNFYPR | CL | K | 0.0052 | 0.0002 | 96 |
| LC (3) | DSTYSLSSTLTLSKADYEK(H) | CL | K | 0.0030 | 0.0002 | 93 |
| LC (3′) | DSTYSLSSTLTLSK(A) | CL | K | 0.0097 | 0.0001 | 99 |
| HC (5) | ****SSASTK(G) | variable/CH1 | K | 1.1074 | 0.0221 | 98 |
| HC (6) | ****SSASTKGPSVFPLAPSSK(S) | variable/CH1 | K | 1.1500 | 0.0198 | 98 |

antibody 3

| | | | | | | |
|---|---|---|---|---|---|---|
| LC (1) | ***********VKG* | CDR-2 | K | 0.0312 | 0.0015 | 95 |
| LC (2′) | ***********VK(G) | CDR-2 | K | 0.0967 | 0.0000 | 100 |
| HC (7) | NQVSLSCAVK(G) | CH3 | K | 0.0789 | 0.0022 | 97 |
| HC (5) | ****SSASTK(G) | variable/CH1 | K | 3.5692 | 0.1145 | 97 |
| HC (6) | ****SSASTKGPSVFPLAPSSK(S) | variable/CH1 | K | 3.1741 | 0.0233 | 99 |

| antibody | PLOD knock-out | titer [μg/mL] |
|---|---|---|
| 1 | no | 5691 |
| | yes | 5856 |
| 2 | no | 3990 |
| | yes | 4796 |
| 3 | no | 4396 |
| | yes | 4587 |

Concomitantly with the increase in titer also the viable cell density increased after the introduction of PLOD gene knockout. The increase is shown by the integral of viable cell density over time (IVCD) in FIGS. 1 to 3 for antibody 1 to 3.

Concomitantly with the increase in titer and viable cell density, also the filterability was improved, i.e. the end pressure in the filtration was reduced. This is shown by the data presented in the following Table and in FIG. 4 (data normalized to respective generated biomass (IVCD)).

| | | initial | starting | end | |
|---|---|---|---|---|---|
| | PLOD | turbidity | pressure | pressure | |
| antibody | knock-out | [NTU] | [bar] | [bar] | load [g/m²] |
| 1 | no | 51.7 | 0.20 | 1.88 | 2280 |
| | yes | 75.5 | 0.24 | 2.02 | 2360 |
| 2 | no | 10.9 | 0.16 | 0.84 | 1600 |
| | yes | 15.7 | 0.19 | 1.72 | 1920 |
| 3 | no | 21.7 | 0.01 | 2.51 | 1662 |
| | yes | 27.7 | 0.20 | 2.10 | 1738 |

Concomitantly with the increase in titer and viable cell density and the improvement in filterability, also the content of the product was improved as determined by size exclusion chromatography (SEC). This is shown in the following Table.

| | PLOD | monomer | LMW | HMW |
|---|---|---|---|---|
| antibody | knock-out | (SEC) [%] | (SEC) [%] | (SEC) [%] |
| 1 | no | 80 | 14 | 6 |
| | yes | 83 | 12.5 | 4.5 |
| 2 | no | 86 | 10 | 4 |
| | yes | 86 | 9 | 5 |
| 3 | no | 75 | 20 | 5 |
| | yes | 77 | 19 | 4 |

***LMW: low molecular weight species content;
HMW: high molecular weight species content

The following examples, sequences and figures are provided to aid the understanding of the present invention, the true scope of which is set forth in the appended claims. It is understood that modifications can be made in the procedures set forth without departing from the spirit of the invention.

Description of the Sequences
SEQ ID NO: 01:
exemplary sequence of an L3 recombinase
recognition sequence:
AAGTCTCC
SEQ ID NO: 02:
exemplary sequence of a 2L recombinase
recognition sequence:
GCATACAT
SEQ ID NO: 03:
exemplary sequence of a LoxFas recombinase
recognition sequence:
TACCTTTC
SEQ ID NO: 04-06:
exemplary variants of human CMV promoter:
SEQ ID NO: 04:
GTTGACATTGATTATTGACTAGTTATTAATAGTAATCAATTACGGGGTCA
TTAGTTCATAGCCCATATATGGAGTTCCGCGTTACATAACTTACGGTAAA
TGGCCCGCCTGGCTGACCGCCCAACGACCCCCGCCCATTGACGTCAATAA
TGACGTATGTTCCCATAGTAACGCCAATAGGGACTTTCCATTGACGTCAA
TGGGTGGAGTATTTACGGTAAACTGCCCACTTGGCAGTACATCAAGTGTA
TCATATGCCAAGTACGCCCCCTATTGACGTCAATGACGGTAAATGGCCCG
CCTGGCATTATGCCCAGTACATGACCTTATGGGACTTTCCTACTTGGCAG
TACATCTACGTATTAGTCATCGCTATTAGCATGGTGATGCGGTTTTGGCA
GTACATCAATGGGCGTGGATAGCGGTTTGACTCACGGGGATTTCCAAGTC
TCCACCCCATTGACGTCAATGGGAGTTTGTTTTGGCACCAAAATCAACGG
GACTTTCCAAAATGTCGTAACAACTCCGCCCCATTGACGCAAATGGGCGG
TAGGCGTGTACGGTGGGAGGTCTATATAAGCAGAGCTCCGTTTAGTGAAC
GTCAGATC
SEQ ID NO: 05:
GTTGACATTGATTATTGACTAGTTATTAATAGTAATCAATTACGGGGTCA
TTAGTTCATAGCCCATATATGGAGTTCCGCGTTACATAACTTACGGTAAA
TGGCCCGCCTGGCTGACCGCCCAACGACCCCCGCCCATTGACGTCAATAA
TGACGTATGTTCCCATAGTAACGCCAATAGGGACTTTCCATTGACGTCAA
TGGGTGGAGTATTTACGGTAAACTGCCCACTTGGCAGTACATCAAGTGTA
TCATATGCCAAGTACGCCCCCTATTGACGTCAATGACGGTAAATGGCCCG
CCTGGCATTATGCCCAGTACATGACCTTATGGGACTTTCCTACTTGGCAG
TACATCTACGTATTAGTCATCGCTATTAGCATGGTGATGCGGTTTTGGCA
GTACATCAATGGGCGTGGATAGCGGTTTGACTCACGGGGATTTCCAAGTC
TCCACCCCATTGACGTCAATGGGAGTTTGTTTTGGCACCAAAATCAACGG
GACTTTCCAAAATGTCGTAACAACTCCGCCCCATTGACGCAAATGGGCGG
TAGGCGTGTACGGTGGGAGGTCTATATAAGCAGAGCTCCGTTTAGTGAAC
GTCAGATCTAGCTCTGGGAGAGGAGCCCAGCACTAGAAGTCGGCGGTGTT
TCCATTCGGTGATCAGCACTGAACACAGAGGAAGCTTGCCGCCACC
SEQ ID NO: 06:
CTGCAGTGAATAATAAAATGTGTGTTTGTCCGAAATACGCGTTTTGAGAT
TTCTGTCGCCGACTAAATTCATGTCGCGCGATAGTGGTGTTTATCGCCGA
TAGAGATGGCGATATTGGAAAAATCGATATTTGAAAATATGGCATATTGA
AAATGTCGCCGATGTGAGTTTCTGTGTAACTGATATCGCCATTTTTCCAA
AAGTGATTTTTGGGCATACGCGATATCTGGCGATAGCGCTTATATCGTTT
ACGGGGGATGGCGATAGACGACTTTGGTGACTTGGGCGATTCTGTGTGTC
GCAAATATCGCAGTTTCGATATAGGTGACAGACGATATGAGGCTATATCG
CCGATAGAGGCGACATCAAGCTGGCACATGGCCAATGCATATCGATCTAT
ACATTGAATCAATATTGGCCATTAGCCATATTATTCATTGGTTATATAGC
ATAAATCAATATTGGCTATTGGCCATTGCATACGTTGTATCCATATCATA
ATATGTACATTTATATTGGCTCATGTCCAACATTACCGCCATGTTGACAT
TGATTATTGACTAGTTATTAATAGTAATCAATTACGGGGTCATTAGTTCA
TAGCCCATATATGGAGTTCCGCGTTACATAACTTACGGTAAATGGCCCGC
CTGGCTGACCGCCCAACGACCCCCGCCCATTGACGTCAATAATGACGTAT
GTTCCCATAGTAACGCCAATAGGGACTTTCCATTGACGTCAATGGGTGGA
GTATTTACGGTAAACTGCCCACTTGGCAGTACATCAAGTGTATCATATGC
CAAGTACGCCCCCTATTGACGTCAATGACGGTAAATGGCCCGCCTGGCAT
TATGCCCAGTACATGACCTTATGGGACTTTCCTACTTGGCAGTACATCTA
CGTATTAGTCATCGCTATTACCATGGTGATGCGGTTTTGGCAGTACATCA
ATGGGCGTGGATAGCGGTTTGACTCACGGGGATTTCCAAGTCTCCACCCC
ATTGACGTCAATGGGAGTTTGTTTTGGCACCAAAATCAACGGGACTTTCC
AAAATGTCGTAACAACTCCGCCCCATTGACGCAAATGGGCGGTAGGCGTG
TACGGTGGGAGGTCTATATAAGCAGAGCTCGTTTAGTGAACCGTCAGATC
GCCTGGAGACGCCATCCACGCTGTTTTGACCTCCATAGAAGACACCGGGA
CCGATCCAGCCTCCGCGGCCGGGAACGGTGCATTGGAACGCGGATTCCCC
GTGCCAAGAGTGACGTAAGTACCGCCTATAGAGTCTATAGGCCCACCCCC
TTGGCTTCTTATGCATGCTATACTGTTTTTGGCTTGGGGTCTATACACCC
CCGCTTCCTCATGTTATAGGTGATGGTATAGCTTAGCCTATAGGTGTGGG
TTATTGACCATTATTGACCACTCCCCTATTGGTGACGATACTTTCCATTA
CTAATCCATAACATGGCTCTTTGCCACAACTCTCTTTATTGGCTATATGC
CAATACACTGTCCTTCAGAGACTGACACGGACTCTGTATTTTTACAGGAT
GGGGTCTCATTTATTATTTACAAATTCACATATACAACACCACCGTCCCC
AGTGCCCGCAGTTTTTATTAAACATAACGTGGGATCTCCACGCGAATCTC
GGGTACGTGTTCCGGACATGGGCTCTTCTCCGGTAGCGGCGGAGCTTCTA
CATCCGAGCCCTGCTCCCATGCCTCCAGCGACTCATGGTCGCTCGGCAGC
TCCTTGCTCCTAACAGTGGAGGCCAGACTTAGGCACAGCACGATGCCCAC
CACCACCAGTGTGCCGCACAAGGCCGTGGCGGTAGGGTATGTGTCTGAAA
ATGAGCTCGGGGAGCGGGCTTGCACCGCTGACGCATTTGGAAGACTTAAG
GCAGCGGCAGAAGAAGATGCAGGCAGCTGAGTTGTTGTGTTCTGATAAGA
GTCAGAGGTAACTCCCGTTGCGGTGCTGTTAACGGTGGAGGGCAGTGTAG
TCTGAGCAGTACTCGTTGCTGCCGCGCGCGCCACCAGACATAATAGCTGA
CAGACTAACAGACTGTTCCTTTCCATGGGTCTTTTCTGCAGTCACCGTCC
TTGACACGGTTTAAACGCCGCCACC
SEQ ID NO: 07:
exemplary SV40 polyadenylation signal sequence:
AACTTGTTTATTGCAGCTTATAATGGTTACAAATAAAGCAATAGCATCAC
AAATTTCACAAATAAAGCATTTTTTTCACCATTCTAGTTGTGGTTTGTCC
AAACTCATCAATGTATCTTATCATGTCTG
SEQ ID NO: 08:
exemplary bGH polyadenylation signal sequence:
TTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGG
GCAGGACAGCAAGGGGGAGGATTGGGAAGACAATAGCAGGCATGCTGGGG
ATGCGGTGGGCTCTATGG
SEQ ID NO: 09:
exemplary hGT terminator sequence:
CAGGATAATATATGGTAGGGTTCATAGCCAGAGTAACCTTTTTTTTTAAT
TTTTATTTTATTTTATTTTT GAG
SEQ ID NO: 10:
exemplary SV40 promoter sequence:
AGTCAGCAACCAGGTGTGGAAAGTCCCCAGGCTCCCCAGCAGGCAGAAGT
ATGCAAAGCATGCATCTCAATTAGTCAGCAACCATAGTCCCGCCCCTAAC
TCCGCCCATCCCGCCCCTAACTCCGCCCAGTTCCGCCCATTCTCCGCCCC
ATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTT
TTGCAAAAAGCTCCCGGGAGCTTGTATATCCATTTTCG
SEQ ID NO: 11:
exemplary GFP nucleic acid sequence:
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGTCCGGACTCAGATCTCGAGCTCAAGCTTCGAAT
TCTGCAGTCGACGGTACCGCGGGCCCGGGATCCACCGGATCTAGATGA
SEQ ID NO: 12-14: SIRT-1 guide RNAs
SEQ ID NO: 12: gRNA_SIRT1_1: TATCATCCAACTCAGGTGGA
SEQ ID NO: 13: gRNA_SIRT1_2: GCAGCATCTCATGATTGGCA
SEQ ID NO: 14: gRNA_SIRT1_3: GCATTCTTGAAGTAACTTCA
SEQ ID NO: 15-16: SIRT-1 PCR primer
SEQ ID NO: 15: SIRT1 for: ATGGCAGTTTTAGACACC
SEQ ID NO: 16: SIRT1 rev: CTTGGAACTCAGACAAGG
SEQ ID NO: 17-19: MYC guide RNAs
SEQ ID NO: 17: gRNA_MYC_1: CTATGACCTCGACTACGACT
SEQ ID NO: 18: gRNA_MYC_2: GGACGCAGCGACCGTCACAT
SEQ ID NO: 19: gRNA MYC_3: CACCATCTCCAGCTGATCCG
SEQ ID NO: 20-21: MYC PCR primer
SEQ ID NO: 20: MYC_for: CACACACACACTTGGAAG
SEQ ID NO: 21: MYC_rev: CTTGATGAAGGTCTCGTC
SEQ ID NO: 22-25: ICAM-1 guide RNAs
SEQ ID NO: 22: gRNA_ICAM1_1: ACCTGCATGGATGCACCCCG
SEQ ID NO: 23: gRNA_ICAM1_2: GCACCGTGCCCACCTCCAGG
SEQ ID NO: 24: gRNA_ICAM1_3: TAACCGCCAGAGAAAGATC
SEQ ID NO: 25: gRNA_ICAM1_4: ACCTGCATGGATGCACCCCG
SEQ ID NO: 26-27: ICAM-1 PCR primer
SEQ ID NO: 26: ICAMI_for: CCAAGCTAGATGATGTGAG
SEQ ID NO: 27: ICAMI_rev: GCCCTACCCTTTTAATAC
SEQ ID NO: 28-32: BAK guide RNAs
SEQ ID NO: 28: gRNA_BAK_1: TACAGCATCTTGGGTCAGGT
SEQ ID NO: 29: gRNA_BAK_2: GTCCATCTCGGGGTTGGCAG
SEQ ID NO: 30: gRNA_BAK_3: AATCTTGGTGAAGAGTTCGT
SEQ ID NO: 31: gRNA_BAK_4: TCATCACAGTCCTGCCTAGG
SEQ ID NO: 32: gRNA_BAK_5: ATGGCGTCTGGACAAGGACC
SEQ ID NO: 33-34: BAK PCR primer
SEQ ID NO: 33: BAK_for: CGTATCTGAGTTCACGAAC
SEQ ID NO: 34: BAK_rev: CCATCAGGAACAAGAGAC
SEQ ID NO: 35-39: BAX guide RNAs
SEQ ID NO: 35: gRNA_BAX_1: ACAGGGGCCTTTTTGCTACA
SEQ ID NO: 36: gRNA_BAX_2: GCTCATCTCCAATTCGCCTG
SEQ ID NO: 37: gRNA_BAX_3: ACGAGAGGTCTTCTTCCGTG
SEQ ID NO: 38: gRNA_BAX_4: GGGTCGGGGGAGCAGCTCGG
SEQ ID NO: 39: gRNA BAX_5: GGGTCCCGAAGTATGAGAGG
SEQ ID NO: 40-41: BAX PCR primer
SEQ ID NO: 40: BAX_for: ATCTTGTCTCCCTCGTAG
SEQ ID NO: 41: BAX_rev: TCCTGGACTTCTCTAACC
SEQ ID NO: 42-44: PLOD1 guide RNAs
SEQ ID NO: 42: gRNA_PLOD1_1: TAAGAGTTCCCGGGGCCCCG
SEQ ID NO: 43: gRNA_PLOD1_2: AACTCATCTACCCCGACCGG
SEQ ID NO: 44: gRNA PLOD1_3: CGCTTGCCATCAGACACCGT
SEQ ID NO: 45-47: PLOD2 guide RNAs
SEQ ID NO: 45: gRNA_PLOD2_1: GTGGCCGGATAAGCGACTCG
SEQ ID NO: 46: gRNA_PLOD2_2: GTTTACCAATGTGCACTACA
SEQ ID NO: 47: gRNA_PLOD2_3: AAACGCTACCTGAATTCTGG
SEQ ID NO: 48-50: PLOD3 guide RNAs
SEQ ID NO: 48: gRNA_PLOD3_1: GATGTTGCTCGAACAGTTGG
SEQ ID NO: 49: gRNA PLOD3_2: GGAGAAATATGCAAACCGGG
SEQ ID NO: 50: gRNA PLOD3_3: CAAATTGCTGGTGATCACCG
SEQ ID NO: 51-53: PLOD artificial substrates
SEQ ID NO: 51: ARGIKGIRGFS
SEQ ID NO: 52: GIKGIKGIKGIK
SEQ ID NO: 53: IKGIKGIKG
SEQ ID NO: 54: HC(1) hydroxylation site-
LTVLSSASTK(G)
SEQ ID NO: 55: HC(2) hydroxylation site-
LTVLSSASTKGPSVFPLAPSSK(S)
SEQ ID NO: 56: HC(3) hydroxylation site-
VTVSSASTK(G)
SEQ ID NO: 57: HC(4) hydroxylation site-
VTVSSASTKGPSVFPLAPSSK(S)
SEQ ID NO: 58: HC(5) hydroxylation site-
SSASTK(G)
SEQ ID NO: 59: HC(6) hydroxylation site-
SSASTKGPSVFPLAPSSK(S)
SEQ ID NO: 60: HC-H hydroxylation site-
NQVSLSCAVK(G)
SEQ ID NO: 61: LC(2) hydroxylation site-
LKSGTASVVCLLNNFYPR
SEQ ID NO: 62: LC(3) hydroxylation site-
DSTYSLSSTLTLSKADYEK(H)
SEQ ID NO: 63: LC(3′) hydroxylation site-
DSTYSLSSTLTLSK(A)
SEQ ID NO: 64: HC-K (1) hydroxylation site-
SSASTK(G)
SEQ ID NO: 65: HC-K (2) hydroxylation site-
SSASTKGPSVFPLAPSSK(S)
SEQ ID NO: 66: background art-
WGQGTLVTVSSASTK

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Integral of viable cell density over time (IVCD) for a cell expressing antibody 1 with (dark green) and without (light green) PLOD1-3 knockout.

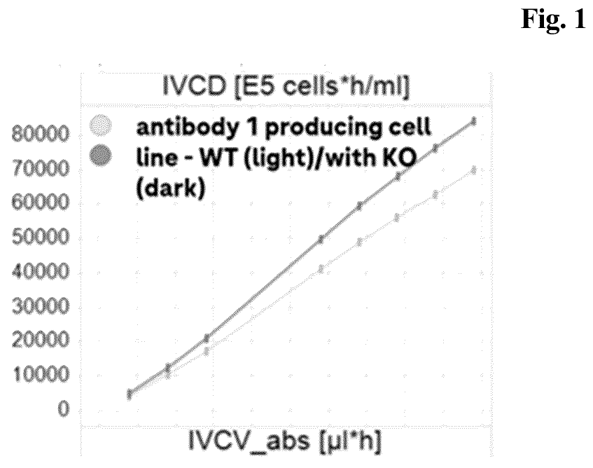

FIG. 2 Integral of viable cell density over time (IVCD) for a cell expressing antibody 2 with (dark blue) and without (light blue) PLOD1-3 knockout.

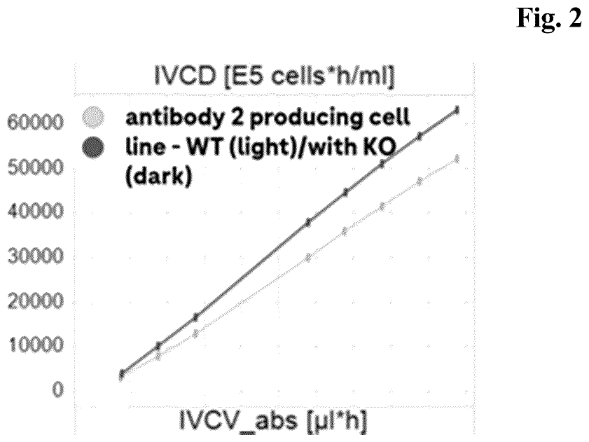

FIG. 3 Integral of viable cell density over time (IVCD) for a cell expressing antibody 3 with (orange) and without (red) PLOD1-3 knock-out.

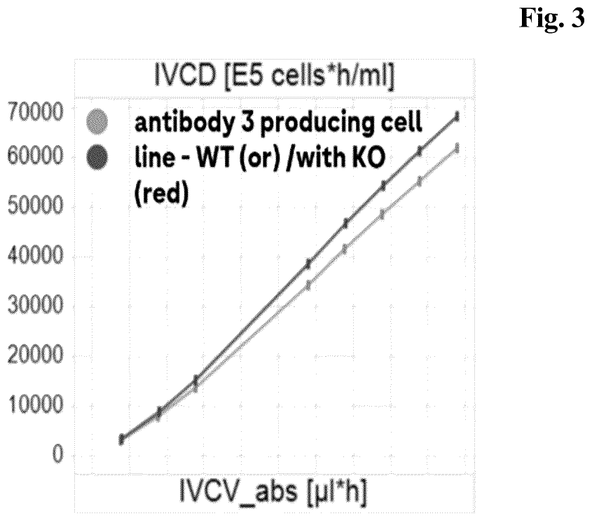

FIG. 4 Change of end pressure in the filtration of antibody 1 to 3 preparations obtained with a cell with or without PLOD1-3 inactivation (data normalized to respective generated biomass (IVCD)).

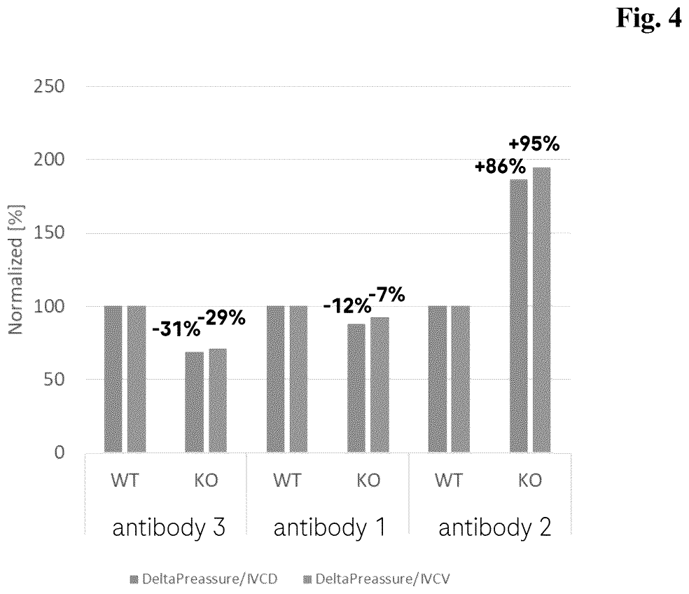

### EXAMPLES

### Example 1—General Techniques

### 1) Recombinant DNA Techniques

Standard methods were used to manipulate DNA as described in Sambrook et al., Molecular Cloning: A Laboratory Manual, Second Edition, Cold Spring Harbor Laboratory Press, Cold Spring Harbor, N.Y, (1989). The molecular biological reagents were used according to the manufacturer's instructions.

### 2) DNA Sequence Determination

DNA sequencing was performed at SequiServe GmbH (Vaterstetten, Germany) or Eurofins Genomics GmbH (Ebersberg, Germany) or Microsynth AG (Balgach, Switzerland).

### 3) DNA and Protein Sequence Analysis and Sequence Data Management

The EMBOSS (European Molecular Biology Open Software Suite) software package and Geneious prime 2021 (Auckland, New Zealand) were used for sequence creation, mapping, analysis, annotation and illustration.

### 4) Gene and Oligonucleotide Synthesis

Desired gene segments were prepared by chemical synthesis at Geneart GmbH (Regensburg, Germany) or Twist Bioscience (San Francisco, USA). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany).

### 5) Reagents

All commercial chemicals, antibodies and kits were used as provided according to the manufacturer's protocol if not stated otherwise.

### 6) Cultivation of TI Host Cell Line

TI CHO host cells were cultivated at 37° C. in a humidified incubator with 85% humidity and 5% CO2. They were cultivated in a proprietary DMEM/F12-based medium containing 300 μg/ml Hygromycin B and 4 μg/ml of a second selection marker. The cells were splitted every 3 or 4 days at a concentration of 0.3×10\hat{ }6 cells/ml in a total volume of 30 ml. For the cultivation 125 ml non-baffle Erlenmeyer shake flasks were used. Cells were shaken at 150 rpm with a shaking amplitude of 5 cm. The cell count was determined with Cedex HiRes Cell Counter (Roche). Cells were kept in culture until they reached an age of 60 days.

### 7) Cloning

### a) General:

Cloning with R-sites depends on DNA sequences next to the gene of interest (GOI) that are equal to sequences lying in following fragments. Like that, assembly of fragments is possible by overlap of the equal sequences and subsequent sealing of nicks in the assembled DNA by a DNA ligase. Therefore, a cloning of the single genes in particular preliminary vectors containing the right R-sites is necessary. After successful cloning of these preliminary vectors the gene of interest flanked by the R-sites is cut out via restriction digest by enzymes cutting directly next to the R-sites. The last step is the assembly of all DNA fragments in one-step. In more detail, a 5′-exonuclease removes the 5′-end of the overlapping regions (R-sites). After that, annealing of the R-sites can take place and a DNA polymerase extends the 3′-end to fill the gaps in the sequence. Finally, the DNA ligase seals the nicks in between the nucleotides. Addition of an assembly master mix containing different enzymes like exonucleases, DNA polymerases and ligases, and subsequent incubation of the reaction mix at 50° C. leads to an assembly of the single fragments to one plasmid. After that, competent E. coli cells are transformed with the plasmid.

For some vectors, a cloning strategy via restriction enzymes was used. By selection of suitable restriction enzymes, the wanted gene of interest can be cut out and afterwards inserted into a different vector by ligation. Therefore, enzymes cutting in a multiple cloning site (MCS) are preferably used and chosen in a smart manner, so that a ligation of the fragments in the correct array can be conducted. If vector and fragment are previously cut with the same restriction enzyme, the sticky ends of fragment and vector fit perfectly together and can be ligated by a DNA ligase, subsequently. After ligation, competent E. coli cells are transformed with the newly generated plasmid.

### b) Cloning Via Restriction Digestion:

For the digest of plasmids with restriction enzymes, the following components were pipetted together on ice:

TABLE
Restriction Digestion Reaction Mix

| component | ng (set point) | μl |
|---|---|---|
| purified DNA | tbd | tbd |
| CutSmart Buffer (10x) | | 5 |
| Restriction Enzyme | | 1 |
| PCR-grade Water | | ad 50 |
| Total | | 50 |

If more enzymes were used in one digestion, 1 μl of each enzyme was used and the volume adjusted by addition of more or less PCR-grade water. All enzymes were selected on the preconditions that they are qualified for the use with CutSmart buffer from new England Biolabs (100% activity) and have the same incubation temperature (all 37° C.).

Incubation was performed using thermomixers or thermal cyclers, allowing incubating the samples at a constant temperature (37° C.). During incubation the samples were not agitated. Incubation time was set at 60 min. Afterwards the samples were directly mixed with loading dye and loaded onto an agarose electrophoresis gel or stored at 4° C./on ice for further use.

A 1% agarose gel was prepared for gel electrophoresis. Therefore, 1.5 g of multi-purpose agarose were weighed into a 125 Erlenmeyer shake flask and filled up with 150 ml TAE-buffer. The mixture was heated up in a microwave oven until the agarose was completely dissolved. 0.5 μg/ml ethidium bromide was added into the agarose solution. Thereafter the gel was cast in a mold. After the agarose was set, the mold was placed into the electrophoresis chamber and the chamber filled with TAE-buffer. Afterwards the samples were loaded. In the first pocket (from the left), an appropriate DNA molecular weight marker was loaded, followed by the samples. The gel was run for around 60 minutes at <130 V. After electrophoresis, the gel was removed from the chamber and analyzed in an UV-Imager.

The target bands were cut and transferred to 1.5 ml Eppendorf tubes. For purification of the gel, the QIAquick Gel Extraction Kit from Qiagen was used according to the manufacturer's instructions. The DNA fragments were stored at −20° C. for further use.

The fragments for the ligation were pipetted together in a molar ratio of 1:2, 1:3 or 1:5 vector to insert, depending on the length of the inserts and the vector-fragments and their correlation to each other. If the fragment that should be inserted into the vector was short, a 1:5-ratio was used. If the insert was longer, a smaller amount of it was used in correlation to the vector. An amount of 50 ng of vector was used in each ligation and the particular amount of insert calculated with NEBioCalculator. For ligation, the T4 DNA ligation kit from NEB was used. An example for the ligation mixture is depicted in the following Table.

TABLE
Ligation Reaction Mix

| Component | ng (set point) | conc. [ng/μl] | μl |
|---|---|---|---|
| T4 DNA Ligase Buffer (10x) | | | 2 |
| Vector DNA (4000 bp) | 50 | 50 | 1 |
| Insert DNA (2000 bp) | 125 | 20 | 6.25 |
| Nuclease-free Water | | | 9.75 |
| T4 Ligase | | | 1 |
| Total | | | 20 |

All components were pipetted together on ice, starting with the mixing of DNA and water, addition of buffer and finally addition of the enzyme. The reaction was gently mixed by pipetting up and down, briefly microfuged and then incubated at room temperature for 10 minutes. After incubation, the T4 ligase was heat inactivated at 65° C. for 10 minutes. The sample was chilled on ice. In a final step, 10-beta competent E. coli cells were transformed with 2 μl of the ligated plasmid (see below).

c) Cloning via R-site assembly:

For assembly, all DNA fragments with the R-sites at each end were pipetted together on ice. An equimolar ratio (0.05 ng) of all fragments was used, as recommended by the manufacturer, when more than 4 fragments are being assembled. One-half of the reaction mix was embodied by NEBuilder HiFi DNA Assembly Master Mix. The total reaction volume was 40 μl and was reached by a fill-up with PCR-clean water. In the following Table, an exemplary pipetting scheme is depicted.

TABLE
Assembly Reaction Mix

| | | pmol | | | |
|---|---|---|---|---|---|
| | | (set | ng | conc. | |
| component | Bp | point) | (set point) | [ng/μl] | μl |
| Insert 1 | 2800 | 0.05 | 88.9 | 21 | 4.23 |
| Insert 2 | 2900 | 0.05 | 90.5 | 35 | 2.59 |
| Insert 3 | 4200 | 0.05 | 131.6 | 35.5 | 3.71 |
| Insert 4 | 3600 | 0.05 | 110.7 | 23 | 4.81 |
| Vector | 4100 | 0.05 | 127.5 | 57.7 | 2.21 |
| NEBuilder HiFi DNA | | | | | 20 |
| Assembly Master Mix | | | | | |
| PCR-clean Water | | | | | 2.45 |
| Total | | | | | 40 |

After setting up the reaction mixture, the tube was incubated in a thermocycler at constantly 50° C. for 60 minutes. After successful assembly, 10-beta competent E. coli bacteria were transformed with 2 μl of the assembled plasmid DNA (see below).

d) Transformation 10-beta competent E. coli cells:

For transformation, the 10-beta competent E. coli cells were thawed on ice. After that, 2 μl of plasmid DNA were pipetted directly into the cell suspension. The tube was flicked and put on ice for 30 minutes. Thereafter, the cells were placed into the 42° C.-warm thermal block and heat-shocked for exactly 30 seconds. Directly afterwards, the cells were chilled on ice for 2 minutes. 950 μl of NEB 10-beta outgrowth medium were added to the cell suspension. The cells were incubated under shaking at 37° C. for one hour. Then, 50-100 μl were pipetted onto a pre-warmed (37° C.) LB-Amp agar plate and spread with a disposable spatula. The plate was incubated overnight at 37° C. Only bacteria, which have successfully incorporated the plasmid, carrying the resistance gene against ampicillin, can grow on these plates. Single colonies were picked the next day and cultured in LB-Amp medium for subsequent plasmid preparation.

### e) Bacterial Culture:

Cultivation of E. coli was done in LB-medium, short for Luria Bertani, which was spiked with 1 ml/L 100 mg/ml ampicillin resulting in an ampicillin concentration of 0.1 mg/ml. For the different plasmid preparation quantities, the following amounts were inoculated with a single bacterial colony.

TABLE
E. coli cultivation volumes

| | volume LB-Amp | incubation |
|---|---|---|
| quantity plasmid preparation | medium [ml] | time [h] |
| Mini-Prep 96-well (EpMotion) | 1.5 | 23 |
| Mini-Prep 15 ml-tube | 3.6 | 23 |
| Maxi-Prep | 200 | 16 |

For Mini-Prep, a 96-well 2 ml deep-well plate was filled with 1.5 ml LB-Amp medium per well. The colonies were picked and the toothpick was tuck in the medium. When all colonies were picked, the plate closed with a sticky air porous membrane. The plate was incubated in a 37° C. incubator at a shaking rate of 200 rpm for 23 hours.

For Mini-Preps a 15 ml-tube (with a ventilated lid) was filled with 3.6 ml LB-Amp medium and equally inoculated with a bacterial colony. The toothpick was not removed but left in the tube during incubation. Like the 96-well plate, the tubes were incubated at 37° C., 200 rpm for 23 hours.

For Maxi-Prep 200 ml of LB-Amp medium were filled into an autoclaved glass 1 L Erlenmeyer flask and inoculated with 1 ml of bacterial day-culture, which was roundabout 5 hours old. The Erlenmeyer flask was closed with a paper plug and incubated at 37° C., 200 rpm for 16 hours.

### f) Plasmid Preparation:

For Mini-Prep, 50 μl of bacterial suspension were transferred into a 1 ml deep-well plate. After that, the bacterial cells were centrifuged down in the plate at 3000 rpm, 4° C. for 5 min. The supernatant was removed and the plate with the bacteria pellets placed into an EpMotion. After approx. 90 minutes, the run was done and the eluted plasmid-DNA could be removed from the EpMotion for further use.

For Mini-Prep, the 15 ml tubes were taken out of the incubator and the 3.6 ml bacterial culture splitted into two 2 ml Eppendorf tubes. The tubes were centrifuged at 6,800×g in a tabletop microcentrifuge for 3 minutes at room temperature. After that, Mini-Prep was performed with the Qiagen QIAprep Spin Miniprep Kit according to the manufacturer's instructions. The plasmid DNA concentration was measured with Nanodrop.

Maxi-Prep was performed using the Macherey-Nagel NucleoBond® Xtra Maxi EF Kit according to the manufacturer's instructions. The DNA concentration was measured with Nanodrop.

### g) Ethanol Precipitation:

The volume of the DNA solution was mixed with the 2.5-fold volume ethanol 100%. The mixture was incubated at −20° C. for 10 min. Then the DNA was centrifuged for 30 min. at 14,000 rpm, 4° C. The supernatant was carefully removed and the pellet washed with 70% ethanol. Again, the tube was centrifuged for 5 min. at 14,000 rpm, 4° C. The supernatant was carefully removed by pipetting and the pellet dried. When the ethanol was evaporated, an appropriate amount of endotoxin-free water was added. The DNA was given time to re-dissolve in the water overnight at 4° C. A small aliquot was taken and the DNA concentration was measured with a Nanodrop device.

### h) Protein Analytic

### CE-SDS

To determine product fragmentation and mispairings, the purified samples were analyzed with capillary electrophoreses (CE). CE on a chip was performed with the LabChip GXII system (PerkinElmer) and the respective protein kits used according to the instructions of the manufacturer. The samples were analyzed in the reduced state.

### SEC

To determine the content of aggregated, fragmented and mis-paired species, the purified samples were analyzed with size exclusion chromatography (SEC).

### LC-MS Peptide Map Procedure

Expressed and protein A affinity chromatography purified antibodies were denatured and reduced with 6 M guanidine and 16 mM DTT at pH 7, 37° C. for 1 hour. The denatured and reduced antibody sample was carboxymethylated using 73 mM IAA-C12 (Fluka), Thereafter the buffer was exchanged on a NAP-5 column (GE Healthcare Life Sciences) to 50 mM TRIS, 2 mM CaCl₂) at pH 7.5 and the antibody digested by Trypsin (Promega) at 37° C. for 1 hour. The digested samples were analyzed by LC-MS/MS. Liquid chromatography was performed on a Waters Acquity UPLC (Waters) with a reversed-phase column Acquity CSH C18, 1.7 μm, 130 Â, 2.1×150 mm (Waters). The aqueous mobile phase (mobile phase A) contained 0.1% (v/v) formic acid (FA) in HPLC grade water. The organic mobile phase (mobile phase B) contained 0.1% (v/v) FA in acetonitrile. The gradient that was utilized in this experiment used a 2-step linear gradient of 1% to 30% mobile phase B from 2 min to 33 min and then to 60% until 42 min followed by an increase to 90% between 42.5 and 44.5 min, a decrease back to 50% between 44.6 min and 50 min followed by a re-equilibration at 1% eluent B from 50 min to 56 min. The column temperature was set to 65° C.

The UPLC was coupled to an Orbitrap Fusion™ mass spectrometer (Thermo Scientific). MS1 spectra were acquired with the Orbitrap mass analyzer with a resolution of 120000 while MS/MS data were acquired in the Orbitrap with a resolution of 50000. The MS/MS event on the Orbitrap was repeated for the topN precursor ions with dynamic exclusion of 4.5 sec enabled.

The resulting MS data were processed by Byos™ & Byonic™ (Protein Metrics Inc.). Manual data interpretation was performed using Byologic™ (Protein Metrics Inc.). The MS/MS Byos™ search settings include a precursor mass tolerance of 5 ppm and a fragment mass tolerance of 20 ppm. The enzyme specificity was set to fully specific with one allowed missed cleavage.

### Example 2—Plasmid Generation

### Expression Cassette Composition

For the expression of an antibody chain, a transcription unit comprising the following functional elements were used:
the immediate early enhancer and promoter from the human cytomegalovirus including intron A,
a human heavy chain immunoglobulin 5′-untranslated region (5′UTR),
a murine immunoglobulin heavy chain signal sequence,
a nucleic acid encoding the respective antibody chain,
the bovine growth hormone polyadenylation sequence (BGH pA), and
optionally the human gastrin terminator (hGT).

Beside the expression unit/cassette including the desired gene to be expressed, the basic/standard mammalian expression plasmid contains:
an origin of replication from the vector pUC18 which allows replication of this plasmid in E. coli, and
a beta-lactamase gene which confers ampicillin resistance in E. coli.
   Front- and back-vector cloning

To construct two-plasmid antibody constructs, antibody HC and LC fragments were cloned into a front vector backbone containing L3 and LoxFas sequences, and a back vector containing LoxFas and 2L sequences and a pac selectable marker. The Cre recombinase plasmid pOG231 (Wong, E. T., et al., Nucl. Acids Res. 33 (2005) e147; O'Gorman, S., et al., Proc. Natl. Acad. Sci. USA 94 (1997) 14602-14607) was used for all RMCE processes.

The cDNAs encoding the respective antibody chains were generated by gene synthesis (Geneart, Life Technologies Inc.). The gene synthesis and the backbone-vectors were digested with HindIII-HF and EcoRI-HF (NEB) at 37° C. for 1 h and separated by agarose gel electrophoresis. The DNA-fragment of the insert and backbone were cut out from the agarose gel and extracted by QIAquick Gel Extraction Kit (Qiagen). The purified insert and backbone fragment was ligated via the Rapid Ligation Kit (Roche) following the manufacturer's protocol with an Insert/Backbone ratio of 3:1. The ligation approach was then transformed in competent E. coli DH5a via heat shock for 30 sec. at 42° C. and incubated for 1 h at 37° C. before they were plated out on agar plates with ampicillin for selection. Plates were incubated at 37° C. overnight.

On the following day clones were picked and incubated overnight at 37° C. under shaking for the Mini or Maxi-Preparation, which was performed with the EpMotion® 5075 (Eppendorf) or with the QIAprep Spin Mini-Prep Kit (Qiagen)/NucleoBond Xtra Maxi EF Kit (Macherey & Nagel), respectively. All constructs were sequenced to ensure the absence of any undesirable mutations (SequiServe GmbH).

In the second cloning step, the previously cloned vectors were digested with KpnI-HF/SalI-HF and SalI-HF/Mfel-HF with the same conditions as for the first cloning. The TI backbone vector was digested with KpnI-HF and Mfel-HF. Separation and extraction was performed as described above. Ligation of the purified insert and backbone was performed using T4 DNA Ligase (NEB) following the manufacturing protocol with an Insert/Insert/Backbone ratio of 1:1:1 overnight at 4° C. and inactivated at 65° C. for 10 min. The following cloning steps were performed as described above.

The cloned plasmids were used for the TI transfection and pool generation.

### Example 3—Cultivation, Transfection, Selection and Single Cell Cloning

TI host cells were propagated in disposable 125 ml vented shake flasks under standard humidified conditions (95% rH, 37° C., and 5% CO2) at a constant agitation rate of 150 rpm in a proprietary DMEM/F12-based medium. Every 3-4 days the cells were seeded in chemically defined medium containing selection marker 1 and selection marker 2 in effective concentrations with a concentration of 3×10\hat{ }5 cells/ml. Density and viability of the cultures were measured with a Cedex HiRes cell counter (F. Hoffmann-La Roche Ltd, Basel, Switzerland).

For stable transfection, equimolar amounts of front and back vector were mixed. Total DNA used per transfection was 30 μg with plasmid ratio 2.5:2.5:1 (front-, back-, Cre plasmid).

Two days prior to transfection TI host cells were seeded in fresh medium with a density of 4×10\hat{ }5 cells/ml. Transfection was performed with the MaxCyte STX electroporation device (MaxCyte Inc., Gaithersburg) using OC-400 electroporation cassettes according to the manufacturer's protocol. 3×10\hat{ }7 cells were transfected with a total of 30 μg nucleic acids, i.e. either with 30 μg plasmid (with a molar ratio of 2.5:2.5:1 of front:back:Cre plasmid)) or with 5 μg Cre mRNA and 25 μg front- and back-vector mixture. After transfection, the cells were seeded in 30 ml medium without selection agents.

On day 5 after seeding the cells were centrifuged and transferred to 80 mL chemically defined medium containing puromycin (selection agent 1) and 1-(2′-deoxy-2′-fluoro-1-beta-D-arabinofuranosyl-5-iodo) uracil (FIAU; selection agent 2) at effective concentrations at 6×10\hat{ }5 cells/ml for selection of recombinant cells. The cells were incubated at 37° C., 150 rpm. 5% CO2, and 85% humidity from this day on without splitting. Cell density and viability of the culture was monitored regularly. When the viability of the culture started to increase again, the concentrations of selection agents 1 and 2 were reduced to about half the amount used before.

In more detail, to promote the recovering of the cells, the selection pressure was reduced if the viability is >40% and the viable cell density (VCD) is >0.5×10\hat{ }6 cells/mL. Therefore, 4×10\hat{ }5 cells/ml were centrifuged and resuspended in 40 ml selection media II (chemically defined medium, ½ selection marker 1 & 2). The cells were incubated with the same conditions as before and also not split.

Ten days after starting selection, the success of Cre mediated cassette exchange was checked by flow cytometry measuring the expression of intracellular GFP and extracellular heterologous polypeptide bound to the cell surface. An APC antibody (allophycocyanin-labeled F(ab′) 2 Fragment goat anti-human IgG) against human antibody light and heavy chain was used for FACS staining. Flow cytometry was performed with a BD FACS Canto II flow cytometer (BD, Heidelberg, Germany). Ten thousand events per sample were measured. Living cells were gated in a plot of forward scatter (FSC) against side scatter (SSC). The live cell gate was defined with non-transfected TI host cells and applied to all samples by employing the FlowJo 10.8.1 EN software (TreeStar, Olten, Switzerland). Fluorescence of GFP was quantified in the FITC channel (excitation at 488 nm, detection at 530 nm). Heterologous polypeptide was measured in the APC channel (excitation at 645 nm, detection at 660 nm). Parental CHO cells, i.e., those cells used for the generation of the TI host cell, were used as a negative control with regard to GFP and heterologous polypeptide expression. Fourteen to twenty-one days after the selection had been started, the viability exceeded 90% and selection was considered as complete.

After selection, the pool of stably transfected cells can be subjected to single-cell cloning by limiting dilution. For this purpose, cells are stained with Cell Tracker Green™ (Thermo Fisher Scientific, Waltham, MA) and plated in 384-well plates with 0.6 cells/well. For single-cell cloning and all further cultivation steps, selection agent 2 is omitted from the medium. Wells containing only one cell are identified by bright field and fluorescence-based plate imaging.

Only wells that contain one cell are further considered. Approximately three weeks after plating colonies are picked from confluent wells and further cultivated in 96-well plates.

### Example 4—FACS Screening

FACS analysis was performed to check the transfection efficiency and the RMCE efficiency of the transfection. 4×10\hat{ }5 cells of the transfected approaches were centrifuged (1200 rpm, 4 min.) and washed twice with 1 mL PBS. After the washing steps with PBS the pellet was resuspended in 400 μL PBS and transferred in FACS tubes (Falcon® Round-Bottom Tubes with cell strainer cap; Corning). The measurement was performed with a FACS Canto II and the data were analyzed by the software FlowJo.

### Example 5—Fed-Batch Cultivation

Fed-batch production cultures were performed in shake flasks or Ambr 15 vessels (Sartorius Stedim) with proprietary chemically defined medium. Cells were seeded at 2×10\hat{ }6 cells/ml on day 0. Cultures received proprietary feed medium on days 3, 7, and 10. Viable cell count (VCC) and percent viability of cells in culture was measured on days 0, 3, 7, 10, and 14 using a Cedex HiRes instrument (Roche Diagnostics GmbH, Mannheim, Germany). Glucose, lactate and product titer concentrations were measured on days 3, 5, 7, 10, 12 and 14 using a Cobas Analyzer (Roche Diagnostics GmbH, Mannheim, Germany). The supernatant was harvested 14 days after start of fed-batch cultivation by centrifugation (10 min, 1000 rpm and 10 min, 4000 rpm) and cleared by filtration (0.22 μm). Day 14 titers were determined using protein A affinity chromatography with UV detection. Product quality was determined by Caliper's LabChip (Caliper Life Sciences).

### Example 6—RNP-Based CRISPR-Cas9 Gene Knockouts

### Material/Resources:

Geneious 2021.2.211.1.5 software for guide and primer design
single clones based on CHO TI host cell line expressing antibody 1, 2 or 3
Gibco TrueCut Cas9 Protein, A45220P, Thermo Fisher TrueCut™ Cas9 Protein v2 (Invitrogen™)
sgRNA (custom designed against target gene, 3 nm chemically modified sgRNA, Synthego)
chemically defined medium (2.5 μg/ml selection agent 2)
DPBS-Dulbecco's Phosphate-Buffered Saline w/o Ca and Mg (Thermo Fisher)
microplate 24 deep well plate (Agilent Technologies, Porvoir science) with cover (self-made)
thin, long RNase, DNase, pyrogen free filter tips for loading OC-100 cassettes (Biozyme)
Hera Safe Hood (Thermo Fisher)
Cedex HiRes Analyzer (Roche Innovatis AG, Bielefeld, Germany)
Liconic Incubator Storex IC
HyClone electroporation buffer
MaxCyte OC-100 cassettes
MaxCyte STX electroporation system

### CRISPR-Cas9 RNP Delivery

RNPs were preassembled by mixing 30 pmol Cas9 with 30 pmol gRNA mix per target gene (equal ratio of each gRNA—see Table below for exemplary genes-specific gRNA sequences) and incubated for 20 minutes at RT. Cells with a concentration between 2-4×10\hat{ }6 cell/mL were centrifuged (3 minutes, 300 g) and washed with 500 μL PBS. Afterwards the cells were again centrifuged (3 minutes at 300 g) and resuspended in 90 μL HyClone electroporation buffer. The pre-incubated RNP mix was added to the cells and incubated for 5 minutes. The cell/RNP solution was then transferred into an OC-100 cuvette and electroporated with program “CHO2” using a MaxCyte electroporation system. Immediately after electroporation, the cell suspension was transferred into a 24 dwell and incubated at 37° C. for 30 minutes. Fresh and pre-warmed medium was added to result in a final cell concentration of 1×10\hat{ }6 cells/mL and incubated at 37° C. with shaking at 350 rpm for cell expansion. For genomic DNA preparation (day 6 or 8), QuickExtract kit (Lucigen) was added to the cells and served as a PCR template. Specific gene amplicons were PCR-amplified using standard Q5 Hot Start Polymerase protocol (NEB) and gene-specific primers that span the gRNA target sites (see sequence listing for the used sequences). The respective amplicon was purified using QIAquick PCR purification kit (Qiagen) and analyzed by Sanger sequencing by Eurofins Genomics GmbH to verify gene inactivation by knockout.

### Example 7—Cell Cultivation

Cells were thawed and cultured in shake flasks for three to four weeks in pre-culture medium with included selection pressure. Cells were passaged every three to four days and expanded in pre-culture medium to reach the volume required for inoculation to non-selective production media. The cells were cultured in Multitron Cell incubators (Infors AG) for the seed and inoculation train phase. Small-scale fermentations were performed in fully controlled, robotic ambr250 fermentation systems from Sartorius Stedim Biotech AG, respectively. All fermentations were controlled for constant dissolved oxygen (DO), temperature, and pH. External pH and pCO2 measurements were monitored using a blood gas analyzer system (pHOx, IUL instruments) or the respective pH and DO ambr analysis module. Viable and total cell densities as well as viability were evaluated using a Cedex HiRes system (Roche). Concentrations of product, substrates, and metabolites/process parameters (glucose, glutamine, glutamate, lactate, ammonium, lactate dehydrogenase, potassium and sodium ions) in the cell culture supernatants were determined utilizing a Cedex BioHT (Roche Innovatis GmbH, Bielefeld, Germany). Amino acids were analyzed with LC-MS system (HILIC-QQQ MS) using the Agilent 1290 HPLC and Ultivo QQQ MS equipment and MassHunter software (Agilent) to observe possible amino acid and substrate limitations and by-product formations.

1. A modified CHO or HEK cell wherein the transcriptional activity of the genes encoding procollagen-lysine,2-oxoglutarate 5-dioxygenase 1 (PLOD1), procollagen-lysine,2-oxoglutarate 5-dioxygenase 2 (PLOD2) and procollagen-lysine,2-oxoglutarate 5-dioxygenase 3 (PLOD3) has been permanently reduced.

2. The modified CHO or HEK cell according to claim 1, wherein further the transcriptional activity of a gene encoding a prolyl hydroxylase has been permanently reduced.

3. The modified CHO or HEK cell according to claim 2, wherein the prolyl hydroxylase is a prolyl 3-hydroxylase (P3H).

4. The modified CHO or HEK cell according to claim 2, wherein the prolyl hydroxylase is prolyl 3-hydroxylase 3 or prolyl 3-hydroxylase 4.

5. The modified CHO or HEK cell according to claim 1, wherein the modified CHO or HEK cell is a modified CHO K1 cell or a modified HEK293 cell.

6. The modified CHO or HEK cell according to claim 1, wherein further the transcriptional activity of one or more or all of the MYC gene or/and the BAX gene, or/and the BAK gene, or/and ICAM-1 gene, or/and the SIRT-1 genes has been permanently reduced.

7. The modified CHO or HEK cell according to claim 1, wherein the modified CHO or HEK cell comprises one or two or more targeted integration landing sites, wherein each targeted integration landing site comprises two or three recombinase recognition sequences, whereby in case of the two or more targeted integration landing sites, the recombinase recognition sequences of each landing site are not compatible with each other.

8. The modified CHO or HEK cell according to claim 7, wherein one of the targeted integration landing sites comprises the recombinase recognition sequences 3L, LoxFas and L2.

9. The modified CHO or HEK cell according to claim 1, wherein at least the reduction of the transcriptional activity of the gene encoding a PLOD has been affected after the stable introduction of one or more nucleic acids encoding a heterologous protein.

10. A method for the recombinant production of a heterologous protein comprising the steps of
a) cultivating a modified CHO or HEK cell according to claim 1 further comprising one or more nucleic acids encoding the heterologous protein in a cultivation medium under conditions suitable for the recombinant expression of the heterologous protein,
b) recovering the heterologous protein from the modified CHO or HEK cell or the cultivation medium,
c) optionally purifying the heterologous protein with one or more chromatography steps, and thereby recombinantly producing the heterologous protein.

11. Use of the permanent reduction of the transcriptional activity of the genes encoding procollagen-lysine,2-oxoglutarate 5-dioxygenase 1 (PLOD1), procollagen-lysine,2-oxoglutarate 5-dioxygenase 2 (PLOD2) and procollagen-lysine,2-oxoglutarate 5-dioxygenase 3 (PLOD3) in a CHO or HEK cell for;
(i) reducing the amount of lysine hydroxylation; or,
(ii) reducing the amount of hydroxy lysine in a heterologous protein produced recombinantly in said CHO or HEK cell; or,
(iii) increasing biomass formation in a cultivation of said CHO or HEK cell; or,
(iv) increasing the titer of a heterologous protein produced recombinantly in said CHO or HEK cell; or,
(v) reducing a heterologous protein side-product formed during cultivation of said CHO or HEK cell that recombinantly expresses said heterologous protein.

12-15. (canceled)


SEQUENCE LISTING (66 sequences)

SEQ ID NO: 1-3 - skipped (3 entries)

SEQ ID NO: 4 - DNA, 608 bp, Human cytomegalovirus
  source 1..608: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatc

SEQ ID NO: 5 - DNA, 696 bp, Human cytomegalovirus
  source 1..696: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatctagctctgggagaggagcccagcactagaagtcggcggtgtttccattcggtgatcagcactgaacacagaggaagcttgccgccacc

SEQ ID NO: 6 - DNA, 2125 bp, Human cytomegalovirus
  source 1..2125: mol_type = other DNA
ctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacggtttaaacgccgccacc

SEQ ID NO: 7 - DNA, 129 bp, Simian virus 40
  source 1..129: mol_type = other DNA
aacttgtttattgcagcttataatggttacaaataaagcaatagcatcacaaatttcacaaataaagcatttttttcaccattctagttgtggtttgtccaaactcatcaatgtatcttatcatgtctg

SEQ ID NO: 8 - DNA, 218 bp, Bos taurus
  source 1..218: mol_type = other DNA
ttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 9 - DNA, 73 bp, Homo sapiens
  source 1..73: mol_type = other DNA
caggataatatatggtagggttcatagccagagtaacctttttttttaatttttattttattttatttttgag

SEQ ID NO: 10 - DNA, 288 bp, Simian virus 40
  source 1..288: mol_type = other DNA
agtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcg

SEQ ID NO: 11 - DNA, 798 bp, synthetic construct
  misc_feature 1..798: note = green fluorescent protein encoding nucleic acid
  source 1..798: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagatga

SEQ ID NO: 12 - DNA, 20 bp, synthetic construct
  misc_feature 1..20: note = gRNA_SIRT1_1
  source 1..20: mol_type = other DNA
tatcatccaactcaggtgga

SEQ ID NO: 13 - DNA, 20 bp, synthetic construct
  misc_feature 1..20: note = gRNA_SIRT1_2
  source 1..20: mol_type = other DNA
gcagcatctcatgattggca

SEQ ID NO: 14 - DNA, 20 bp, synthetic construct
  misc_feature 1..20: note = gRNA_SIRT1_3
  source 1..20: mol_type = other DNA
gcattcttgaagtaacttca

SEQ ID NO: 15 - DNA, 18 bp, synthetic construct
  misc_feature 1..18: note = SIRT1_for
  source 1..18: mol_type = other DNA
atggcagttttagacacc

SEQ ID NO: 16 - DNA, 18 bp, synthetic construct
  misc_feature 1..18: note = SIRT1_rev
  source 1..18: mol_type = other DNA
cttggaactcagacaagg

SEQ ID NO: 17 - DNA, 20 bp, synthetic construct
  misc_feature 1..20: note = gRNA_MYC_1
  source 1..20: mol_type = other DNA
ctatgacctcgactacgact

SEQ ID NO: 18 - DNA, 20 bp, synthetic construct
  misc_feature 1..20: note = gRNA_MYC_2
  source 1..20: mol_type = other DNA
ggacgcagcgaccgtcacat

SEQ ID NO: 19 - DNA, 20 bp, synthetic construct
  misc_feature 1..20: note = gRNA_MYC_3
  source 1..20: mol_type = other DNA
caccatctccagctgatccg

SEQ ID NO: 20 - DNA, 18 bp, synthetic construct
  misc_feature 1..18: note = MYC_for
  source 1..18: mol_type = other DNA
cacacacacacttggaag

SEQ ID NO: 21 - DNA, 18 bp, synthetic construct
  misc_feature 1..18: note = MYC_rev
  source 1..18: mol_type = other DNA
cttgatgaaggtctcgtc

SEQ ID NO: 22 - DNA, 20 bp, synthetic construct
  misc_feature 1..20: note = gRNA_ICAM1_1
  source 1..20: mol_type = other DNA
acctgcatggatgcaccccg

SEQ ID NO: 23 - DNA, 20 bp, synthetic construct
  misc_feature 1..20: note = gRNA_ICAM1_2
  source 1..20: mol_type = other DNA
gcaccgtgcccacctccagg

SEQ ID NO: 24 - DNA, 19 bp, synthetic construct
  misc_feature 1..19: note = gRNA_ICAM1_3
  source 1..19: mol_type = other DNA
taaccgccagagaaagatc

SEQ ID NO: 25 - DNA, 20 bp, synthetic construct
  misc_feature 1..20: note = gRNA_ICAM1_4
  source 1..20: mol_type = other DNA
acctgcatggatgcaccccg

SEQ ID NO: 26 - DNA, 19 bp, synthetic construct
  misc_feature 1..19: note = ICAM1_for
  source 1..19: mol_type = other DNA
ccaagctagatgatgtgag

SEQ ID NO: 27 - DNA, 18 bp, synthetic construct
  misc_feature 1..18: note = ICAM1_rev
  source 1..18: mol_type = other DNA
gccctacccttttaatac

SEQ ID NO: 28 - DNA, 20 bp, synthetic construct
  misc_feature 1..20: note = gRNA_BAK_1
  source 1..20: mol_type = other DNA
tacagcatcttgggtcaggt

SEQ ID NO: 29 - DNA, 20 bp, synthetic construct
  misc_feature 1..20: note = gRNA_BAK_2
  source 1..20: mol_type = other DNA
gtccatctcggggttggcag

SEQ ID NO: 30 - DNA, 20 bp, synthetic construct
  misc_feature 1..20: note = gRNA_BAK_3
  source 1..20: mol_type = other DNA
aatcttggtgaagagttcgt

SEQ ID NO: 31 - DNA, 20 bp, synthetic construct
  misc_feature 1..20: note = gRNA_BAK_4
  source 1..20: mol_type = other DNA
tcatcacagtcctgcctagg

SEQ ID NO: 32 - DNA, 20 bp, synthetic construct
  misc_feature 1..20: note = gRNA_BAK_5
  source 1..20: mol_type = other DNA
atggcgtctggacaaggacc

SEQ ID NO: 33 - DNA, 19 bp, synthetic construct
  misc_feature 1..19: note = BAK_for
  source 1..19: mol_type = other DNA
cgtatctgagttcacgaac

SEQ ID NO: 34 - DNA, 18 bp, synthetic construct
  misc_feature 1..18: note = BAK_rev
  source 1..18: mol_type = other DNA
ccatcaggaacaagagac

SEQ ID NO: 35 - DNA, 20 bp, synthetic construct
  misc_feature 1..20: note = gRNA_BAX_1
  source 1..20: mol_type = other DNA
acaggggcctttttgctaca

SEQ ID NO: 36 - DNA, 20 bp, synthetic construct
  misc_feature 1..20: note = gRNA_BAX_2
  source 1..20: mol_type = other DNA
gctcatctccaattcgcctg

SEQ ID NO: 37 - DNA, 20 bp, synthetic construct
  misc_feature 1..20: note = gRNA_BAX_3
  source 1..20: mol_type = other DNA
acgagaggtcttcttccgtg

SEQ ID NO: 38 - DNA, 20 bp, synthetic construct
  misc_feature 1..20: note = gRNA_BAX_4
  source 1..20: mol_type = other DNA
gggtcgggggagcagctcgg

SEQ ID NO: 39 - DNA, 20 bp, synthetic construct
  misc_feature 1..20: note = gRNA_BAX_5
  source 1..20: mol_type = other DNA
gggtcccgaagtatgagagg

SEQ ID NO: 40 - DNA, 18 bp, synthetic construct
  misc_feature 1..18: note = BAX_for
  source 1..18: mol_type = other DNA
atcttgtctccctcgtag

SEQ ID NO: 41 - DNA, 18 bp, synthetic construct
  misc_feature 1..18: note = BAX_rev
  source 1..18: mol_type = other DNA
tcctggacttctctaacc

SEQ ID NO: 42 - DNA, 20 bp, synthetic construct
  misc_feature 1..20: note = gRNA_PLOD1_1
  source 1..20: mol_type = other DNA
taagagttcccggggccccg

SEQ ID NO: 43 - DNA, 20 bp, synthetic construct
  misc_feature 1..20: note = gRNA_PLOD1_2
  source 1..20: mol_type = other DNA
aactcatctaccccgaccgg

SEQ ID NO: 44 - DNA, 20 bp, synthetic construct
  misc_feature 1..20: note = gRNA_PLOD1_3
  source 1..20: mol_type = other DNA
cgcttgccatcagacaccgt

SEQ ID NO: 45 - DNA, 20 bp, synthetic construct
  misc_feature 1..20: note = gRNA_PLOD2_1
  source 1..20: mol_type = other DNA
gtggccggataagcgactcg

SEQ ID NO: 46 - DNA, 20 bp, synthetic construct
  misc_feature 1..20: note = gRNA_PLOD2_2
  source 1..20: mol_type = other DNA
gtttaccaatgtgcactaca

SEQ ID NO: 47 - DNA, 20 bp, synthetic construct
  misc_feature 1..20: note = gRNA_PLOD2_3
  source 1..20: mol_type = other DNA
aaacgctacctgaattctgg

SEQ ID NO: 48 - DNA, 20 bp, synthetic construct
  misc_feature 1..20: note = gRNA_PLOD3_1
  source 1..20: mol_type = other DNA
gatgttgctcgaacagttgg

SEQ ID NO: 49 - DNA, 20 bp, synthetic construct
  misc_feature 1..20: note = gRNA_PLOD3_2
  source 1..20: mol_type = other DNA
ggagaaatatgcaaaccggg

SEQ ID NO: 50 - DNA, 20 bp, synthetic construct
  misc_feature 1..20: note = gRNA_PLOD3_3
  source 1..20: mol_type = other DNA
caaattgctggtgatcaccg

SEQ ID NO: 51 - protein, 11 aa, synthetic construct
  REGION 1..11: note = PLOD artificial substrate 1
ARGIKGIRGFS

SEQ ID NO: 52 - protein, 12 aa, synthetic construct
  REGION 1..12: note = PLOD artificial substrate 2
GIKGIKGIKGIK

SEQ ID NO: 53 - protein, 9 aa, synthetic construct
  REGION 1..9: note = PLOD artificial substrate 3
IKGIKGIKG

SEQ ID NO: 54 - protein, 11 aa, Homo sapiens
LTVLSSASTKG

SEQ ID NO: 55 - protein, 23 aa, Homo sapiens
LTVLSSASTKGPSVFPLAPSSKS

SEQ ID NO: 56 - protein, 10 aa, Homo sapiens
VTVSSASTKG

SEQ ID NO: 57 - protein, 22 aa, Homo sapiens
VTVSSASTKGPSVFPLAPSSKS

SEQ ID NO: 58 - protein, 7 aa, Homo sapiens
SSASTKG

SEQ ID NO: 59 - protein, 19 aa, Homo sapiens
SSASTKGPSVFPLAPSSKS

SEQ ID NO: 60 - protein, 11 aa, Homo sapiens
NQVSLSCAVKG

SEQ ID NO: 61 - protein, 18 aa, Homo sapiens
LKSGTASVVCLLNNFYPR

SEQ ID NO: 62 - protein, 20 aa, Homo sapiens
DSTYSLSSTLTLSKADYEKH

SEQ ID NO: 63 - protein, 15 aa, Homo sapiens
DSTYSLSSTLTLSKA

SEQ ID NO: 64 - protein, 7 aa, Homo sapiens
SSASTKG

SEQ ID NO: 65 - protein, 19 aa, Homo sapiens
SSASTKGPSVFPLAPSSKS

SEQ ID NO: 66 - protein, 15 aa, synthetic construct
  REGION 1..15: note = background art
WGQGTLVTVSSASTK